SEQUENCE LISTING (101 sequences)

SEQ ID NO: 1 - protein, 78 aa, Artificial Sequence
  OLP HIV clade B consensus sequence
EKIRLRPGGKKKYKLKHIVWASRELERFAVNPGLLETSEGCRQILGQLQPSLQTGSEELKSLYNTVATLYCVHQKIEV

SEQ ID NO: 2 - protein, 14 aa, Artificial Sequence
  OLP HIV clade B consensus sequence
KAFSPEVIPMFSAL

SEQ ID NO: 3 - protein, 11 aa, Artificial Sequence
  OLP HIV clade B consensus sequence
GHQAAMQMLKE

SEQ ID NO: 4 - protein, 60 aa, Artificial Sequence
  OLP HIV clade B consensus sequence
IAPGQMREPRGSDIAGTTSTLQEQIGWMTNNPPIPVGEIYKRWIILGLNKIVRMYSPTSI

SEQ ID NO: 5 - protein, 14 aa, Artificial Sequence
  OLP HIV clade B consensus sequence
YVDRFYKTLRAEQA

SEQ ID NO: 6 - protein, 15 aa, Artificial Sequence
  OLP HIV clade B consensus sequence
ACQGVGGPGHKARVL

SEQ ID NO: 7 - protein, 27 aa, Artificial Sequence
  OLP HIV clade B consensus sequence
CTERQANFLGKIWPSHKGRPGNFLQSR

SEQ ID NO: 8 - protein, 55 aa, Artificial Sequence
  OLP HIV clade B consensus sequence
KMIGGIGGFIKVRQYDQILIEICGHKAIGTVLVGPTPVNIIGRNLLTQIGCTLNF

SEQ ID NO: 9 - protein, 17 aa, Artificial Sequence
  OLP HIV clade B consensus sequence
LVEICTELEKEGKISKI

SEQ ID NO: 10 - protein, 55 aa, Artificial Sequence
  OLP HIV clade B consensus sequence
LRWGFTTPDKKHQKEPPFLWMGYELHPDKWTVQPIVLPEKDSWTVNDIQKLVGKL

SEQ ID NO: 11 - protein, 34 aa, Artificial Sequence
  OLP HIV clade B consensus sequence
ILKEPVHGVYYDPSKDLIAEIQKQGQGQWTYQIY

SEQ ID NO: 12 - protein, 34 aa, Artificial sequence
  OLP HIV clade B consensus sequence
TKELQKQITKIQNFRVYYRDSRDPLWKGPAKLLW

SEQ ID NO: 13 - protein, 17 aa, Artificial Sequence
  OLP HIV clade B consensus sequence
KIIRDYGKQMAGDDCVA

SEQ ID NO: 14 - protein, 26 aa, Artificial Sequence
  OLP HIV clade B consensus sequence
VKHHMYISKKAKGWFYRHHYESTHPR

SEQ ID NO: 15 - protein, 19 aa, Artificial Sequence
  OLP HIV clade B consensus sequence
VTKLTEDRWNKPQKTKGHR

SEQ ID NO: 16 - protein, 13 aa, Artificial Sequence
  OLP HIV clade B consensus sequence
AWLEAQEEEEVGF

SEQ ID NO: 17 - protein, 23 aa, Artificial Sequence
  OLP HIV clade B consensus sequence
EKIRLRPGGKKKYKLKHIVWASR

SEQ ID NO: 18 - protein, 22 aa, Artificial Sequence
  OLP HIV clade B consensus sequence
KLKHIVWASRELERFAVNPGLL

SEQ ID NO: 19 - protein, 23 aa, Artificial Sequence
  OLP HIV clade B consensus sequence
ERFAVNPGLLETSEGCRQILGQL

SEQ ID NO: 20 - protein, 24 aa, Artificial Sequence
  OLP HIV clade B consensus sequence
EGCRQILGQLQPSLQTGSEELKSL

SEQ ID NO: 21 - protein, 26 aa, Artificial Sequence
  OLP HIV clade B consensus sequence
QTGSEELKSLYNTVATLYCVHQRIEV

SEQ ID NO: 22 - protein, 14 aa, Artificial Sequence
  OLP HIV clade B consensus sequence
KAFSPEVIPMFSAL

SEQ ID NO: 23 - protein, 11 aa, Artificial Sequence
  OLP HIV clade B consensus sequence
GHQAAMQMLKE

SEQ ID NO: 24 - protein, 21 aa, Artificial Sequence
  OLP HIV clade B consensus sequence
IAPGQMREPRGSDIAGTTSTL

SEQ ID NO: 25 - protein, 23 aa, Artificial Sequence
  OLP HIV clade B consensus sequence
SDIAGTTSTLQEQIGWMTNNPPI

SEQ ID NO: 26 - protein, 24 aa, Artificial Sequence
  OLP HIV clade B consensus sequence
IGWMTNNPPIPVGEIYKRWIILGL

SEQ ID NO: 27 - protein, 22 aa, Artificial Sequence
  OLP HIV clade B consensus sequence
IYKRWIILGLNKIVRMYSPTSI

SEQ ID NO: 28 - protein, 14 aa, Artificial Sequence
  OLP HIV clade B consensus sequence
YVDRFYKTLRAEQA

SEQ ID NO: 29 - protein, 15 aa, Artificial Sequence
  OLP HIV clade B consensus sequence
ACQGVGGPGHKARVL

SEQ ID NO: 30 - protein, 27 aa, Artificial Sequence
  OLP HIV clade B consensus sequence
CTERQANFLGKIWPSHKGRPGNFLQSR

SEQ ID NO: 31 - protein, 26 aa, Artificial Sequence
  OLP HIV clade B consensus sequence
KMIGGIGGFIKVRQYDQILIEICGHK

SEQ ID NO: 32 - protein, 24 aa, Artificial Sequence
  OLP HIV clade B consensus sequence
QILIEICGHKAIGTVLVGPTPVNI

SEQ ID NO: 33 - protein, 25 aa, Artificial Sequence
  OLP HIV clade B consensus sequence
VLVGPTPVNIIGRNLLTQIGCTLNF

SEQ ID NO: 34 - protein, 17 aa, Artificial Sequence
  OLP HIV clade B consensus sequence
LVEICTEMEKEGKISKI

SEQ ID NO: 35 - protein, 25 aa, Artificial Sequence
  OLP HIV clade B consensus sequence
LRWGFTTPDKKHQKEPPFLWMGYEL

SEQ ID NO: 36 - protein, 25 aa, Artificial Sequence
  OLP HIV clade B consensus sequence
PPFLWMGYELHPDKWTVQPIVLPEK

SEQ ID NO: 37 - protein, 25 aa, Artificial Sequence
  OLP HIV clade B consensus sequence
TVQPIVLPEKDSWTVNDIQKLVGKL

SEQ ID NO: 38 - protein, 23 aa, Artificial Sequence
  OLP HIV clade B consensus sequence
ILKEPVHGVYYDPSKDLIAEIQK

SEQ ID NO: 39 - protein, 21 aa, Artificial Sequence
  OLP HIV clade B consensus sequence
SKDLIAEIQKQGQGQWTYQIY

SEQ ID NO: 40 - protein, 22 aa, Artificial Sequence
  OLP HIV clade B consensus sequence
TKELQKQITKIQNFRVYYRDSR

SEQ ID NO: 41 - protein, 22 aa, Artificial Sequence
  OLP HIV clade B consensus sequence
NFRVYYRDSRDPLWKGPAKLLW

SEQ ID NO: 42 - protein, 17 aa, Artificial Sequence
  OLP HIV clade B consensus sequence
KIIRDYGKQMAGDDCVA

SEQ ID NO: 43 - protein, 26 aa, Artificial Sequence
  OLP HIV clade B consensus sequence
VKHHMYISKKAKGWFYRHHYESTHPR

SEQ ID NO: 44 - protein, 19 aa, Artificial Sequence
  OLP HIV clade B consensus sequence
VTKLTEDRWNKPQKTKGHR

SEQ ID NO: 45 - protein, 13 aa, Artificial Sequence
  OLP HIV clade B consensus sequence
AWLEAQEEEEVGF

SEQ ID NO: 46 - protein, 17 aa, Homo sapiens
MWLQSLLLLGTVACSIS

SEQ ID NO: 47 - protein, 18 aa, Artificial Sequence
  human GM-CSF signal peptide + V
MWLQSLLLLGTVACSISV

SEQ ID NO: 48 - protein, 9 aa, Artificial Sequence
  Flag-tag
DYKDDDDKL

SEQ ID NO: 49 - protein, 556 aa, Artificial Sequence
  HIV immunogen comprising OLPs
MWLQSLLLLGTVACSISVEKIRLRPGGKKKYKLKHIVWASRELERFAVNPGLLETSEGCRQILGQLQPSLQTGSEELKSLYNTVATLYCVHQKIEVAAAKAFSPEVIPMFSALAAAGHQAAMQMLKEAAAIAPGQMREPRGSDIAGTTSTLQEQIGWMTNNPPIPVGEIYKRWIILGLNKIVRMYSPTSIAAAYVDRFYKTLRAEQAAACQGVGGPGHKARVLAAACTERQANFLGKIWPSHKGRPGNFLQSRAAAKMIGGIGGFIKVRQYDQILIEICGHKAIGTVLVGPTPVNIIGRNLLTQIGCTLNFAALVEICTEMEKEGKISKIAAALRWGFTTPDKKHQKEPPFLWMGYELHPDKWTVQPIVLPEKDSWTVNDIQKLVGKLAAAILKEPVHGVYYDPSKDLIAEIQKQGQGQWTYQIYAAATKELQKQITKIQNFRVYYRDSRDPLWKGPAKLLWAAAKIIRDYGKQMAGDDCVAAAVKHHMYISKKAKGWFYRHHYESTHPRAAAVTKLTEDRWNKPQKTKGHRAAAWLEAQEEEEVGFDYKDDDDKL

SEQ ID NO: 50 - DNA, 1674 bp, Artificial Sequence
  polynucleotide encoding immunogen comprising HIV OLPs
atgtggctccagagcctgctactcctggggacggtggcctgcagcatctcggtcgagaagatccggctgcggccaggcggaaagaagaagtacaagctgaagcacatcgtctgggcctcgagggagctggagcggttcgcggtgaacccgggacttctggagacgtcggaggggtgcaggcagatcctcggccagctgcagccctctctgcaaacggggtctgaggagctgaagagcctgtacaacacggtggcgaccctctactgcgtccaccagaagatcgaggtggcagcggccaaggcgttctcgccggaggtcatccccatgttctcggcgctggcagctgccggacaccaggccgcgatgcagatgctgaaggaggccgctgcgatcgcaccgggccagatgagggagccacgcggttccgacatcgcgggaaccacctcgacgctccaggagcagatcggatggatgacgaacaacccgccaatcccggtcggggagatctacaagcggtggatcatcctcgggctgaacaagatcgtccggatgtacagcccgacgtcgatcgctgcggcatacgttgaccggttctacaagaccctgagggccgagcaggcagcggcctgccagggggtcggtggaccagggcacaaggcccgagtgctcgcggccgcatgcacggagcggcaggcgaacttcctggggaagatctggccgtcgcacaagggccgaccgggaaacttcctccagtctcgcgcagcggctaagatgatcggaggcatcggaggcttcatcaaagtccgtcagtacgaccagatcctcatcgagatctgcgggcacaaggcgatcggaaccgtgctcgtcggcccaacgcccgtgaacatcatcggccgcaacctgttaacgcagatcggctgcaccctcaacttcgccgcactagtggagatctgcacggagatggagaaggagggcaagatatcgaagatcgcggcagctctgaggtggggcttcaccacgccggacaagaagcaccagaaggagccgccattcctgtggatgggatacgagctgcacccggacaagtggaccgtgcagcccatcgtcctgccggagaaggactcgtggacggtgaacgacatccagaagctcgtggggaagctggcggcagccatcctcaaggagcccgtccacggggtgtactacgacccctctaaggacctgatcgcggagatccagaagcaggggcagggtcagtggacctaccagatctacgcagcagcaaccaaggagctgcagaagcagatcacgaagatccagaacttccgcgtatactaccgcgactcgcgggaccccctgtggaagggccctgcgaagcttctctgggcagccgcgaagatcatccgggactacggcaagcagatggcgggcgacgactgcgtggccgcagcggtgaagcaccatatgtacatctcgaagaaggcgaagggctggttctacagacaccactacgagtccacccaccccagggcagctgcggtgacgaagctgacggaggaccggtggaacaagccccagaagacgaagggtcaccgggcggctgcatggctggaggctcaggaggaggaggaggtgggcttcgattacaaggacgatgacgacaagctgtgataa

SEQ ID NO: 51 - DNA, 60 bp, Artificial Sequence
  198HPlus primer
gtcaccgggcggctgcatggctggaggctcaggaggaggaggaggtgggcttctgataag

SEQ ID NO: 52 - DNA, 59 bp, Artificial Sequence
  298HMinus primer
aattcttatcagaagcccacctcctcctcctcctgagcctccagccatgcagccgcccg

SEQ ID NO: 53 - protein, 18 aa, Artificial Sequence
  OLP clade B consensus sequence
EKIRLRPGGKKKYKLKHI

SEQ ID NO: 54 - protein, 15 aa, Artificial Sequence
  OLP clade B consensus sequence
ASRELERFAVNPGLL

SEQ ID NO: 55 - protein, 17 aa, Artificial Sequence
  OLP clade B consensus sequence
ERFAVNPGLLETSEGCR

SEQ ID NO: 56 - protein, 17 aa, Artificial Sequence
  OLP clade B consensus sequence
QLQPSLQTGSEELRSLY

SEQ ID NO: 57 - protein, 18 aa, Artificial Sequence
  OLP clade B consensus sequence
SLYNTVATLYCVHQRIEV

SEQ ID NO: 58 - protein, 17 aa, Artificial Sequence
  OLP clade B consensus sequence
AFSPEVIPMFSALSEGA

SEQ ID NO: 59 - protein, 15 aa, Artificial Sequence
  OLP clade B consensus sequence
IAPGQMREPRGSDIA

SEQ ID NO: 60 - protein, 18 aa, Artificial Sequence
  OLP clade B consensus sequence
STLQEQIGWMTNNPPIPV

SEQ ID NO: 61 - protein, 18 aa, Artificial Sequence
  OLP clade B consensus sequence
ACQGVGGPGHKARVLAEA

SEQ ID NO: 62 - protein, 18 aa, Artificial Sequence
  OLP clade B consensus sequence
GKIWPSHKGRPGNFLQSR

SEQ ID NO: 63 - protein, 18 aa, Artificial Sequence
  OLP clade B consensus sequence
WLEAQEEEEVGFPVRPQV

SEQ ID NO: 64 - protein, 18 aa, Artificial Sequence
  OLP clade B consensus sequence
EVGFPVRPQVPLRPMTYK

SEQ ID NO: 65 - protein, 18 aa, Artificial Sequence
  OLP clade B consensus sequence
NYTPGPGIRYPLTFGWCF

SEQ ID NO: 66 - protein, 15 aa, Artificial Sequence
  OLP clade B consensus sequence
RYPLTFGWCFKLVPV

SEQ ID NO: 67 - protein, 17 aa, Artificial Sequence
  OLP clade B consensus sequence
SLHGMDDPEKEVLVWKF

SEQ ID NO: 68 - protein, 18 aa, Artificial Sequence
  OLP clade B consensus sequence
KMIGGIGGFIKVRQYDQI

SEQ ID NO: 69 - protein, 18 aa, Artificial Sequence
  OLP clade B consensus sequence
FIKVRQYDQILIEICGHK

SEQ ID NO: 70 - protein, 17 aa, Artificial Sequence
  OLP clade B consensus sequence
QILIEICGHKAIGTVLV

SEQ ID NO: 71 - protein, 18 aa, Artificial Sequence
  OLP clade B consensus sequence
LVGPTPVNIIGRNLLTQI

SEQ ID NO: 72 - protein, 17 aa, Artificial Sequence
  OLP clade B consensus sequence
LVEICTEMEKEGKISKI

SEQ ID NO: 73 - protein, 18 aa, Artificial Sequence
  OLP clade B consensus sequence
LDVGDAYFSVPLDKDFRK

SEQ ID NO: 74 - protein, 18 aa, Artificial Sequence
  OLP clade B consensus sequence
LRWGFTTPDKKHQKEPPF

SEQ ID NO: 75 - protein, 18 aa, Artificial Sequence
  OLP clade B consensus sequence
DKKHQKEPPFLWMGYELH

SEQ ID NO: 76 - protein, 15 aa, Artificial Sequence
  OLP clade B consensus sequence
EIQKQGQGQWTYQIY

SEQ ID NO: 77 - protein, 18 aa, Artificial Sequence
  OLP clade B consensus sequence
PPLVKLWYQLEKEPIVGA

SEQ ID NO: 78 - protein, 15 aa, Artificial Sequence
  OLP clade B consensus sequence
IHLALQDSGLEVNIV

SEQ ID NO: 79 - protein, 18 aa, Artificial Sequence
  OLP clade B consensus sequence
VYLAWVPAHKGIGGNEQV

SEQ ID NO: 80 - protein, 16 aa, Artificial Sequence
  OLP clade B consensus sequence
SAGIRKVLFLDGIDKA

SEQ ID NO: 81 - protein, 18 aa, Artificial Sequence
  OLP clade B consensus sequence
TKELQKQITKIQNFRVYY

SEQ ID NO: 82 - protein, 18 aa, Artificial Sequence
  OLP clade B consensus sequence
TKIQNFRVYYRDSRDPLW

SEQ ID NO: 83 - protein, 18 aa, Artificial Sequence
  OLP clade B consensus sequence
YYRDSRDPLWKGPAKLLW

SEQ ID NO: 84 - protein, 17 aa, Artificial Sequence
  OLP clade B consensus sequence
KIIRDYGKQMAGDDCVA

SEQ ID NO: 85 - protein, 18 aa, Artificial Sequence
  OLP clade B consensus sequence
GPQREPYNEWTLELLEEL

SEQ ID NO: 86 - protein, 18 aa, Artificial Sequence
  OLP clade B consensus sequence
DLNNNTNTTSSSGEKMEK

SEQ ID NO: 87 - protein, 18 aa, Artificial Sequence
  OLP clade B consensus sequence
IRDKVQKEYALFYKLDVV

SEQ ID NO: 88 - protein, 18 aa, Artificial Sequence
  OLP clade B consensus sequence
YRLISCNTSVITQACPKV

SEQ ID NO: 89 - protein, 18 aa, Artificial Sequence
  OLP clade B consensus sequence
SVITQACPKVSFEPIPIH

SEQ ID NO: 90 - protein, 16 aa, Artificial Sequence
  OLP clade B consensus sequence
TNVSTVQCTHGIRPVV

SEQ ID NO: 91 - protein, 18 aa, Artificial Sequence
  OLP clade B consensus sequence
VAPTKAKRRVVQREKRAV

SEQ ID NO: 92 - protein, 18 aa, Artificial Sequence
  OLP clade B consensus sequence
VIEVVQRACRAILHIPRR

SEQ ID NO: 93 - protein, 18 aa, Artificial Sequence
  OLP clade B consensus sequence
VKHHMYISGKAKGWFYRH

SEQ ID NO: 94 - protein, 18 aa, Artificial Sequence
  OLP clade B consensus sequence
GKAKGWFYRHHYESTHPR

SEQ ID NO: 95 - protein, 18 aa, Artificial Sequence
  OLP clade B consensus sequence
TKLTEDRWNKPQKTKGHR

SEQ ID NO: 96 - protein, 6 aa, Artificial Sequence
  Tag sequence
AHGHRP

SEQ ID NO: 97 - protein, 14 aa, Artificial Sequence
  Tag sequence
PIHDHDHPHLVIHS

SEQ ID NO: 98 - protein, 7 aa, Artificial Sequence
  Tag sequence
  misc_feature (5)..(6): Xaa can be any naturally occurring amino acid
GMTCXXC

SEQ ID NO: 99 - protein, 530 aa, Artificial Sequence
  HIV immunogen comprising OLPs
EKIRLRPGGKKKYKLKHIVWASRELERFAVNPGLLETSEGCRQILGQLQPSLQTGSEELKSLYNTVATLYCVHQKIEVAAAKAFSPEVIPMFSALAAAGHQAAMQMLKEAAAIAPGQMREPRGSDIAGTTSTLQEQIGWMTNNPPIPVGEIYKRWIILGLNKIVRMYSPTSIAAAYVDRFYKTLRAEQAAACQGVGGPGHKARVLAAACTERQANFLGKIWPSHKGRPGNFLQSRAAAKMIGGIGGFIKVRQYDQILIEICGHKAIGTVLVGPTPVNIIGRNLLTQIGCTLNFAAALVEICTEMEKEGKISKIAAALRWGFTTPDKKHQKEPPFLWMGYELHPDKWTVQPIVLPEKDSWTVNDIQKLVGKLAAAILKEPVHGVYYDPSKDLIAEIQKQGQGQWTYQIYAAATKELQKQITKIQNFRVYYRDSRDPLWKGPAKLLWAAAKIIRDYGKQMAGDDCVAAAVKHHMYISKKAKGWFYRHHYESTHPRAAAVTKLTEDRWNKPQKTKGHRAAAWLEAQEEEEVGF

SEQ ID NO: 100 - DNA, 1587 bp, Artificial Sequence
  polynucleotide encoding immunogen comprising HIV OLPs
gagaagatccggctgcggccaggcggaaagaagaagtacaagctgaagcacatcgtctgggcctcgagggagctggagcggttcgcggtgaacccgggacttctggagacgtcggaggggtgcaggcagatcctcggccagctgcagccctctctgcaaacggggtctgaggagctgaagagcctgtacaacacggtggcgaccctctactgcgtccaccagaagatcgaggtggcagcggccaaggcgttctcgccggaggtcatccccatgttctcggcgctggcagctgccggacaccaggccgcgatgcagatgctgaaggaggccgctgcgatcgcaccgggccagatgagggagccacgcggttccgacatcgcgggaaccacctcgacgctccaggagcagatcggatggatgacgaacaacccgccaatcccggtcggggagatctacaagcggtggatcatcctcgggctgaacaagatcgtccggatgtacagcccgacgtcgatcgctgcggcatacgttgaccggttctacaagaccctgagggccgagcaggcagcggcctgccagggggtcggtggaccagggcacaaggcccgagtgctcgcggccgcatgcacggagcggcaggcgaacttcctggggaagatctggccgtcgcacaagggccgaccgggaaacttcctccagtctcgcgcagcggctaagatgatcggaggcatcggaggcttcatcaaagtccgtcagtacgaccagatcctcatcgagatctgcgggcacaaggcgatcggaaccgtgctcgtcggcccaacgcccgtgaacatcatcggccgcaacctgttaacgcagatcggctgcaccctcaacttcgccgcactagtggagatctgcacggagatggagaaggagggcaagatatcgaagatcgcggcagctctgaggtggggcttcaccacgccggacaagaagcaccagaaggagccgccattcctgtggatgggatacgagctgcacccggacaagtggaccgtgcagcccatcgtcctgccggagaaggactcgtggacggtgaacgacatccagaagctcgtggggaagctggcggcagccatcctcaaggagcccgtccacggggtgtactacgacccctctaaggacctgatcgcggagatccagaagcaggggcagggtcagtggacctaccagatctacgcagcagcaaccaaggagctgcagaagcagatcacgaagatccagaacttccgcgtatactaccgcgactcgcgggaccccctgtggaagggccctgcgaagcttctctgggcagccgcgaagatcatccgggactacggcaagcagatggcgggcgacgactgcgtggccgcagcggtgaagcaccatatgtacatctcgaagaaggcgaagggctggttctacagacaccactacgagtccacccaccccagggcagctgcggtgacgaagctgacggaggaccggtggaacaagccccagaagacgaagggtcaccgggcggctgcatggctggaggctcaggaggaggaggaggtgggcttc

SEQ ID NO: 101 - DNA, 1593 bp, Artificial Sequence
  Polynucleotide encoding immunogen comprising HIV OLPs
gagaagatccgcctgcgccccggcggcaagaaaaagtacaagctgaagcacatcgtgtgggcctcccgcgagctggagcgcttcgccgtgaaccccggcctgctggagacctccgagggctgccgccagatcctgggccagctgcagccctccctgcagaccggctccgaggagctgaagtccctgtacaacaccgtggccaccctgtactgcgtgcaccagaagatcgaggtggccgccgccaaggccttctcccccgaggtgatccccatgttctccgccctggccgccgccggccaccaggccgccatgcagatgctgaaggaggccgccgccatcgcccccggccagatgcgcgagccccgcggctccgacatcgccggcaccacctccaccctgcaggagcagatcggctggatgaccaacaacccccccatccccgtgggcgagatctacaagcgctggatcatcctgggcctgaacaagatcgtgcgcatgtactcccccacctccatcgccgccgcctacgtggaccgcttctacaagaccctgcgcgccgagcaggccgccgcctgccagggcgtgggcggccccggccacaaggcccgcgtgctggccgccgcctgcaccgagcgccaggccaacttcctgggcaagatctggccctcccacaagggccgccccggcaacttcctgcagtcccgcgccgccgccaagatgatcggcggcatcggcggcttcatcaaggtgcgccagtacgaccagatcctgatcgagatctgcggccacaaggccatcggcaccgtgctggtgggccccacccccgtgaacatcatcggccgcaacctgctgacccagatcggctgcaccctgaacttcgccgccctggtggagatctgcaccgagatggagaaggagggcaagatctccaagatcgccgccgccctgcgctggggcttcaccacccccgacaagaagcaccagaaggagccccccttcctgtggatgggctacgagctgcaccccgacaagtggaccgtgcagcccatcgtgctgcccgagaaggactcctggaccgtgaacgacatccagaagctggtgggcaagctggccgccgccatcctgaaggagcccgtgcacggcgtgtactacgacccctccaaggacctgatcgccgagatccagaagcagggccagggccagtggacctaccagatctacgccgccgccaccaaggagctgcagaagcagatcaccaagatccagaacttccgcgtgtactaccgcgactcccgcgaccccctgtggaagggccccgccaagctgctgtgggccgccgccaagatcatccgcgactacggcaagcagatggccggcgacgactgcgtggccgccgccgtgaagcaccacatgtacatctccaagaaggccaagggctggttctaccgccaccactacgagtccacccacccccgcgccgccgccgtgaccaagctgaccgaggaccgctggaacaagccccagaagaccaagggccaccgcgccgccgcctggctggaggcccaggaggaggaagaggtgggcttctgatag

(19) United States
(12) Patent Application Publication
(10) Pub. No.: US 2022/0226459 A1
(43) Pub. Date: Jul. 21, 2022

(54) DOSAGE REGIMENS FOR VACCINES

(71) Applicant: AELIX THERAPEUTICS, S.L., BARCELONA (ES)

(72) Inventors: CHRISTIAN BRANDER, TIANA (ES); BEATRIZ MOTHE PUJADAS, TIANA (ES); IAN MCGOWAN, BARCELONA (ES)

(21) Appl. No.: 17/612,426

(22) PCT Filed: May 21, 2020
(86) PCT No.: PCT/IB2020/054864

§ 371 Date: Nov. 18, 2021

Related U.S. Application Data
(60) Provisional application No. 62/851,546, filed on May 22, 2019.

(51) Int. Cl. A61K 39/12; A61P 31/18; C12N 7/00

(52) U.S. Cl. CPC A61K 39/12; A61K 2039/5256; C12N 7/00; A61P 31/18

(57) ABSTRACT

The present invention relates to immunogenic therapies for the treatment or prevention of a human immunodeficiency virus (HIV) infection or a disease associated with an HIV infection.

### CROSS-REFERENCE TO RELATED APPLICATIONS

This application claims priority to U.S. Provisional Patent Application No. 62/851,546, filed on May 22, 2019, which is incorporated by reference herein in its entirety.

### REFERENCE TO SEQUENCE LISTING SUBMITTED ELECTRONICALLY

The content of the electronically submitted Sequence Listing (“3834_006PC01_Seqlisting_ST25”; Size: 45,681 bytes; and Date of Creation: May 12, 2020) filed with the application is incorporated herein by reference in its entirety.

### FIELD OF THE INVENTION

The present invention relates to immunogenic therapies for the treatment or prevention of a human immunodeficiency virus (HIV) infection or a disease associated with an HIV infection.

### BACKGROUND OF THE INVENTION

Increased access to highly active combination antiretroviral therapy (cART) has resulted in a dramatic decrease in morbidity and mortality associated with infection by HIV. However, despite having new classes of antiretroviral drugs, currently available cART regimens are not able to eradicate HIV from the body. Consequently, cART cessation in participants maintaining undetectable viral load is followed by a fast rebound in viremia. Moltó et al., AIDS Res Hum Retroviruses. 2004; 20(12):1283-8; El-Sadr et al., N Engl J Med. 2006; 355(22):2283-96. This reflects the inability of the standard cART in eliminating a viral reservoir formed by latently infected cells in which the integrated provirus remains quiescent and stable in early stages of infection, and the inability of the immune response to effectively contain viral rebound after treatment interruption.

Even though cART results in control of the viral load (thus preventing the development of AIDS and virus transmission), it has several shortcomings:

1. Not curative: cART are treatments for life. If a person stops the treatment, even for a short period of time, the viral load rebounds to initial levels within 2-4 weeks, making this person infective again.

2. Adherence issues: 30 to 50% of patients are not able to control the viral load, because they don't follow the treatment regime rigorously enough. This has much to do with psychological stress—living with HIV with no cure in sight affects a patient's quality of life—and even without that, all patients are inconvenienced by their treatment routines, to varying degrees (“pill fatigue”).

3. Resistance: HIV can develop resistance to cART.

4. Side-effects: Because of the high long-term toxicity of cART, patients suffer from serious adverse events, such as cardiovascular diseases, dyslipidemias, hypertension, diabetes, osteoporosis, and kidney diseases.

5. High and permanent cost: Treating a patient with cART costs about €20.000 per year, while the total cost for the health system during the patient life time is calculated to be €400.000.

6. Social stigma: The stigma surrounding HIV makes people reluctant to get tested, or to disclose their HIV status; it also limits their access to available HIV treatment.

Multiple strategies have been evaluated to try to achieve an optimal control of HIV infection in the absence of cART. These have included early treatment initiation within the first 6 months after HIV acquisition, cART intensification, immunotherapies including interleukin administrations (IL-2, IL-7, IL-10, IL-12, and IL-15), treatment with cyclosporine, mycophenolate, hydroxyurea, thalidomide, passive administration of antibodies, etc. and a wide range of therapeutic vaccines designed to expand the response mediated by cytotoxic T lymphocytes. Buzón et al., Nat Med. 2010; 16(4):460-5; Autran et al., AIDS. 2008; 22(11):1313-22; Schooley et al., J Infect Dis. 2010; 202(5):705-16; Harrer et al., Vaccine. 2014; 32(22):2657-65.

Minimal clinical effect has been observed after a vaccination strategy with an autologous dendritic-cell vaccination approach, which was able to demonstrate transient 1 log reduction in the viral setpoint of vaccinated compared to unvaccinated patients after discontinuation of treatment. Garcia et al., Sci Transl Med. 2013; 5(166):166ra2. In addition, recent data from a pilot study suggests that re-education of T cells towards conserved regions of HIV by therapeutic vaccines in early treated patients (<6 months of HIV acquisition) may contribute to durable HIV control in a considerable proportion of participants after treatment cessation. Mothe et al., CROI 2017, 119LB. Both sets of results set the stage for improved therapeutic vaccine concepts.

An important cause of a therapeutic vaccine's failure is the composition of the antigen insert (immunogen) expressed in the vectors, the combinations thereof used for the administration of the vaccine, and in the dosing regimen of the vaccine components to be administered. In particular, the inclusion of whole HIV proteins as antigens limits the immunogenic effect of the vaccine towards a nonspecific cytotoxic T lymphocyte (CTL) expansion: a CTL response pattern which, in natural HIV infection, has been shown ineffective in controlling viral replication in most individuals. Mothe et al., J Transl Med. 2011; 9(1):208; Pereyra et al., J Virol. 2014; 88(22):12937-48.

In this regard, there is a need to improve the immunogen design by selecting viral sequences able to induce T cell responses which are more beneficial to the host. Letourneau et al., PLoS One. 2007; 2(10):e984; Rolland et al., PLoS Pathogens. 2007; 3:1551-5; Mothe et al., J Transl Med. 2015; 13(1):60.

Moreover, HIV-1 infection induces strong and broadly directed HLA class I and class II restricted T-cell responses, for which some specific epitopes and restricting HLA alleles have been associated with relative in vivo virus control or lack thereof. Brander et al., Curr Opin Immunol. 2006; 18(4):430-7; Zuñiga et al., Virol. 2006; 80(6):3122-5; Frahm et al., Nat Immunol. 2006; 7(2):173-8. Among these, CD8+ CTL responses to HIV-1 Gag have most consistently been associated with reduced viral loads in both HIV-1 Glade B- and C-infected cohorts. Zuñiga et al., Virol. 2006; 80(6):3122-5; Kiepiela et al., Nat Med. 2007; 13(1):46-53. CD4+ T-cell responses to Gag have also been associated with relative HIV-1 control. Ranasinghe et al., J Virol. 2012; 86(1):277-83; Ranasinghe et al., Nat Med. 2013; 19(7):930-3. In addition, the elevated level of conservation of Gag across viral isolates and the severe fitness reductions caused by CTL escape variants may provide Gag-specific T-cell responses with a particular advantage.

At the same time, it is also clear that not all Gag-specific responses exert the same antiviral activity, suggesting that a rational selection of Gag components could help focus vaccine induced responses onto the most protective targets. The same likely applies for all other viral proteins as well, as they may contain some regions that are of particular value for inclusion in a vaccine while other regions or proteins may induce less useful T cell responses. As such, effective vaccine design should likely aim to induce broad and evenly distributed responses to conserved and vulnerable sites of the virus while avoiding the induction of responses to regions that can be highly immunogenic but that may act as potential “decoy” targets and divert responses away from more relevant targets. Rolland et al., PLoS Pathogens. 2007; 3:1551-5; Kulkarni et al., PLoS One. 2013; 8(3):e60245; Kulkarni et al., PLoS One. 2014; 9(1):e86254; Dinges et al., J Virol. 2010; 84(9):4461-8; Kunwar et al., PLoS One. 2013; 8(5):e64405; Niu et al., Vaccine. 2011; 29(11):2110-9.

The failure of various T-cell vaccine candidates expressing entire HIV-1 proteins in large human clinical trials and data from post-trial analyses suggests a sieve effect on the infecting viral strains and indicates there is a need to improve vaccine immunogen design. Buchbinder et al., Lancet. 2008; 372(9653):1881-93; Rerks-Ngarm et al., N Engl J Med. 2009; 361(23):2209-20; Hammer et al., N Engl J Med. 2013; 369(22):2083-92; Rolland et al., Nat Med. 2011; 17(3):366-71.

### SUMMARY OF THE INVENTION

In one aspect, the present invention relates to a method of treating or preventing a human immunodeficiency virus (HIV) infection or a disease associated with an HIV infection in a subject in need thereof, comprising (a) administering to the subject 1 to 10 administrations of a DNA vector encoding an immunogenic polypeptide, followed by 1 to 10 administrations of a first viral vector encoding the immunogenic polypeptide; and (b) administering to the subject 1 to 10 administrations of a second viral vector encoding the immunogenic polypeptide; wherein the immunogenic polypeptide comprises:

(i) a sequence having at least 90% identity to the sequence of SEQ ID NO:1,

(ii) a sequence having at least 90% identity to the sequence of SEQ ID NO:2,

(iii) a sequence having at least 90% identity to the sequence of SEQ ID NO:3,

(iv) a sequence having at least 90% identity to the sequence of SEQ ID NO:4,

(v) a sequence having at least 90% identity to the sequence of SEQ ID NO:5,

(vi) a sequence having at least 90% identity to the sequence of SEQ ID NO:6,

(vii) a sequence having at least 90% identity to the sequence of SEQ ID NO:7,

(viii) a sequence having at least 90% identity to the sequence of SEQ ID NO:8,

(ix) a sequence having at least 90% identity to the sequence of SEQ ID NO:9,

(x) a sequence having at least 90% identity to the sequence of SEQ ID NO:10,

(xi) a sequence having at least 90% identity to the sequence of SEQ ID NO:11,

(xii) a sequence having at least 90% identity to the sequence of SEQ ID NO:12,

(xiii) a sequence having at least 90% identity to the sequence of SEQ ID NO:13,

(xiv) a sequence having at least 90% identity to the sequence of SEQ ID NO:14,

(xv) a sequence having at least 90% identity to the sequence of SEQ ID NO:15, and

(xvi) a sequence having at least 90% identity to the sequence of SEQ ID NO:16.

In another aspect, the present invention relates to a method of treating or preventing a HIV infection or a disease associated with an HIV infection in a subject in need thereof, comprising (a) administering to the subject 1 to 5 administrations of a first viral vector encoding the immunogenic polypeptide; and (b) administering to the subject 1 to 5 administrations of a second viral vector encoding the immunogenic polypeptide; wherein the immunogenic polypeptide comprises:

(i) a sequence having at least 90% identity to the sequence of SEQ ID NO:1,

(ii) a sequence having at least 90% identity to the sequence of SEQ ID NO:2,

(iii) a sequence having at least 90% identity to the sequence of SEQ ID NO:3,

(iv) a sequence having at least 90% identity to the sequence of SEQ ID NO:4,

(v) a sequence having at least 90% identity to the sequence of SEQ ID NO:5,

(vi) a sequence having at least 90% identity to the sequence of SEQ ID NO:6,

(vii) a sequence having at least 90% identity to the sequence of SEQ ID NO:7,

(viii) a sequence having at least 90% identity to the sequence of SEQ ID NO:8,

(ix) a sequence having at least 90% identity to the sequence of SEQ ID NO:9,

(x) a sequence having at least 90% identity to the sequence of SEQ ID NO:10,

(xi) a sequence having at least 90% identity to the sequence of SEQ ID NO:11,

(xii) a sequence having at least 90% identity to the sequence of SEQ ID NO:12,

(xiii) a sequence having at least 90% identity to the sequence of SEQ ID NO:13,

(xiv) a sequence having at least 90% identity to the sequence of SEQ ID NO:14,

(xv) a sequence having at least 90% identity to the sequence of SEQ ID NO:15, and

(xvi) a sequence having at least 90% identity to the sequence of SEQ ID NO:16.

### BRIEF DESCRIPTION OF THE DRAWINGS

FIG. 1 shows the median and interquartile range of the total breadth (FIG. 1A) and total magnitude (FIG. 1B) of HTI responses to ChAdOx1.HTI and ChAdNou68.HTI vaccinated mice. Significant differences in the Mann-Whitney test are indicated by *0.05>p>0.01.

FIG. 2 shows scatterplots with 95% confidence interval of the ELISpot results for female mice (FIGS. 2A-2B), male mice (FIGS. 2C-2D, and both female and male mice (FIGS. 2E-2F) at day 134 (D134), normalized with their respective negative controls.

FIGS. 2A, 2C and 2E contain all mice (N=5 per sex, per group, except for group 1c male (N=4)). FIGS. 2B, 2D and 2F exclude mice with a number of spots >20 in negative controls (N=5 except for 1b female (N=3), 1b male (N=4), 2b female and male (N=4), 3a female (N=4)). Groups with lighter shaded spots are groups containing mice with a number of spots in negative control >20.

FIG. 3 shows scatterplots with 95% confidence interval of the ELISpot results for 7 treatment groups of mice (FIG. 3A-3G, respectively, for Groups 1-7 (G1-G7)). Group 1: PBS. Group 2: DNA.HTI (PB108)+MVA.HTI (0010915). Group 3: PBS DNA.HTI (PB125). Group 4: PBS+DNA.HTI (PB136). Group 5: DNA.HTI (PB125)+MVA.HTI (0010216). Group 6: DNA.HTI (PB136)+MVA.HTI (0020518). Group 7: PBS+ChAdOx2.HTI (H.0003). N=6 per group.

FIG. 4 shows the IFN-γ response in mice that received buffer (Group 1) or ChAdOx1.HTI (Group 2). Results for male mice are shown in FIG. 4A, and results for female mice are shown in FIG. 4B. There was an increase of around 10 fold for both males and females in the IFN-γ response for all Group 2 (ChAdOx1.HTI) animals compared to Group 1 (Buffer) animals in wells treated with peptide #23. Wells treated with peptide #101 resulted in a small increase of around 3 fold in the IFN-γ for all Group 2 animals compared to Group 1 animals for both males and females. Positive control wells (ConA and CD3) and negative control (media) control wells were also evaluated.

FIG. 5A shows the number of positive pools from C57BL/6 females given the indicated treatments.

FIG. 5B shows the number of INFγ spot forming colonies per 10⁶ splenocytes from C57BL/6 females given the indicated treatments.

FIG. 6A shows the number of positive pools from BalbC males and females given the indicated treatments.

FIG. 6B shows the number of INFγ spot forming colonies per 10⁶ splenocytes from BalbC males and females given the indicated treatments.

FIG. 7 shows a comparison of the different prime-boost strategies in FIGS. 5-6.

FIG. 8 shows the induction of HIV-1 specific T-cell responses by the BCG.HTI+ChAd.HTI prime-boost regimen in BALB/c mice. Adult mice (7 weeks old, n=8/group) were immunized with either BCG.HTI2auxo.int (id) and boosted with ChAdOx1.HTI (10⁹ vp, im), with BCG.wt (id) and boosted with ChAdOx1.HTI (10⁹ vp, im), with ChAdOx1.HTI (10⁹ vp, im) (Group C), or left unimmunized (Group D). The treatment groups and immunization schedule are shown in FIG. 8A. Two weeks post-boost, mice were sacrificed and splenocytes were isolated for ELISpot analysis (FIG. 8B). The total magnitude of HIV-1 specific SFCs/10⁶ splenocytes was calculated as sums of the 17 HTI peptide pools, the color-coding represents the HIV-1 gene location. Data are presented in FIGS. 8A-8B as group means and error bars represent standard deviation of the total sum of SFC/10⁶ splenocytes. Statistics were performed using parametric one-way ANOVA. IFN-γ spot forming cells (SFC)/10⁶ are also shown in response to HTI-derived peptide pools representing HIV-1 Gag (FIG. 8C), HIV-1 Pol (FIG. 8D) and Nef, Vif and Tuberculin PPD (FIG. 8E). The data are presented as medians of group responses above the threshold. Statistics were performed using the non-parametric Kruskal-Wallis test, *p<0.05, **p<0.01, ***p<0.001.

FIG. 9 shows the differential recognition of peptide pools in BCG.HTI+ChAd.HTI immunized BALB/c mice. Adult mice (7-weeks-old, n=8/group) were immunized with either 10⁵ cfu of BCG.HTI2auxo.int (id) and boosted with ChAdOx1.HTI (10⁹ vp, im) after 5 weeks (Group A), or with 10⁶ BCG.wt (id) and boosted with ChAdOx1.HTI (10⁹ vp, im) after 5 weeks (Group B), or only immunized with ChAdOx1.HTI (10⁹ vp, im) at week 5 (Group C), or left unimmunized (Group D). Two weeks post-boost, mice were sacrificed and splenocytes were isolated for ELISpot analysis and the numbers of reactive peptide pools (total n peptide pools=17) were compared for each mouse.

FIG. 10 shows a study design to test the clinical efficacy of a prime/boost strategy of the present invention in HIV-1 positive individuals.

FIG. 11 shows a design of the intervention for a study to test the clinical efficacy of a prime/boost strategy of the present invention in HIV-1 positive individuals.

### DETAILED DESCRIPTION OF THE INVENTION

The present invention is directed to methods of treating or preventing a human immunodeficiency virus (HIV) infection or a disease associated with an HIV infection using an HIV immunogen termed HTI.

### Definitions

The term “adjuvant”, as used herein, refers to an immunological agent that modifies the effect of an immunogen, while having few if any direct effects when administered by itself. It is often included in vaccines to enhance the recipient's immune response to a supplied antigen, while keeping the injected foreign material to a minimum. Adjuvants are added to vaccines to stimulate the immune system's response to the target antigen, but do not in themselves confer immunity. Non-limiting examples of useful adjuvants include mineral salts, polynucleotides, polyarginines, ISCOMs, saponins, monophosphoryl lipid A, imiquimod, CCR-5 inhibitors, toxins, polyphosphazenes, cytokines, immunoregulatory proteins, immunostimulatory fusion proteins, co-stimulatory molecules, and combinations thereof. Mineral salts include, but are not limited to, AIK(SO₄)₂, A1Na(SO₄)₂, AlNH(SO₄)₂, silica, alum, Al(OH)₃, Ca₃(PO₄)₂, kaolin, or carbon. Useful immunostimulatory polynucleotides include, but are not limited to, CpG oligonucleotides with or without immune stimulating complexes (ISCOMs), CpG oligonucleotides with or without polyarginine, poly IC or poly AU acids. Toxins include cholera toxin. Saponins include, but are not limited to, QS21, QS17 or QS7. An example of a useful immunostimulatory fusion protein is the fusion protein of IL-2 with the Fc fragment of immunoglobulin. Useful immunoregulatory molecules include, but are not limited to, CD40L and CD1a ligand. Cytokines useful as adjuvants include, but are not limited to, IL-1, IL-2, IL-4, GMCSF, IL-12, IL-15, IGF-1, IFNα, IFN-β, and interferon gamma. Also, examples of are muramyl dipeptides, N-acetyl-muramyl-L-threonyl-D-isoglutamine (thr-DMP), N-acetyl-nornuramyl-L-alanyl-D-isoglutamine (CGP 11687, also referred to as nor-MDP), N-acetylmuramyul-L-alanyl-D-isoglutaminyl-L-alanine-2-(1′-dipalmitoyl-sn-glycero-3-hydroxphosphoryloxy)-ethylamine (CGP 19835A, also referred to as MTP-PE), RIBI (MPL+TDM+CWS) in a 2 percent squalene/TWEEN® 80 emulsion, lipopolysaccharides and its various derivatives, including lipid A, Freund's Complete Adjuvant (FCA), Freund's Incomplete Adjuvants, Merck Adjuvant 65, polynucleotides (e.g., poly IC and poly AU acids), wax D from Mycobacterium tuberculosis, substances found in Corynebacterium parvum, Bordetella pertussis, and members of the genus Brucella, Titermax, Quil A, ALUN, Lipid A derivatives, choleratoxin derivatives, HSP derivatives, LPS derivatives, synthetic peptide matrixes or GMDP, Montanide ISA-51 and QS-21, CpG oligonucleotide, poly I:C, and GMCSF. See Osol A., Ed., Remington's Pharmaceutical Sciences (Mack Publishing Co., Easton, Pa., US, 1980, pp. 1324-1341), Hunter R, U.S. Pat. No. 5,554,372, and Jager E, Knuth A, WO1997028816. Combinations of adjuvants can also be used.

The term “AIDS”, as used herein, refers to the symptomatic phase of HIV infection, and includes both Acquired Immune Deficiency Syndrome (commonly known as AIDS) and “ARC,” or AIDS-Related Complex. Adler et al., Brit. Med. J. 1987; 294: 1145-1147. The immunological and clinical manifestations of AIDS are well known in the art and include, for example, opportunistic infections and cancers resulting from immune deficiency.

The term “amino acid linker”, as used herein, refers to an amino acid sequence other than that appearing at a particular position in the natural protein and is generally designed to be flexible or to interpose a structure, such as an α-helix, between two protein moieties. A linker is also referred to as a spacer. The linker is typically non-antigenic and can be of essentially any length (e.g., 2, 3, 4, 5, 6, 7, 8, 9, 10, 11, 12, 13, 14, 15, 16, 17, 18, 19, 20, or more amino acids). The linker may also be a location or sequence where the cellular antigen processing machinery can initiate the degradation of the immunogenic polypeptide without destroying potent T cell epitopes).

The term “codon optimized”, as used herein, relates to the alteration of codons in nucleic acid molecules to reflect the typical codon usage of the host organism without altering the polypeptide encoded by the DNA, to improve expression. A plethora of methods and software tools for codon optimization have been reported previously. Narum et al., Infect. Immun. 2001; 69(12):7250-7253, Outchkourov et al., Protein Expr. Purif. 2002; 24(1):18-24, Feng L, et al., Biochemistry 2000; 39(50):15399-15409, and Humphreys et al., Protein Expr. Purif. 2000; 20(2):252-2.

The terms “comprising” or “comprises”, as used herein, disclose also “consisting of” according to the generally accepted patent practice.

The expression “disease associated with a HIV infection”, as used herein, includes a state in which the subject has developed AIDS, but also includes a state in which the subject infected with HIV has not shown any sign or symptom of the disease. Thus, the vaccine of the invention when administered to a subject that has no clinical signs of the infection can have a preventive activity, since they can prevent the onset of the disease. The immunogenic compositions are capable of preventing or slowing the infection and destruction of healthy CD4+ T cells in such a subject. It also refers to the prevention and slowing the onset of symptoms of the acquired immunodeficiency disease such as extreme low CD4+ T cell count and repeated infections by opportunistic pathogens such as Mycobacteria spp., Pneumocystis carinii, and Pneumocystis cryptococcus. Beneficial or desired clinical results include, but are not limited to, an increase in absolute naive CD4+ T cell count (range 10-3520), an increase in the percentage of CD4+ T cell over total circulating immune cells (range 1-50 percent), and/or an increase in CD4+ T cell count as a percentage of normal CD4+ T cell count in an uninfected subject (range 1-161 percent).

The terms “variant” and “fragment”, as used herein, refer to a polypeptide derived from any of SEQ ID NOs:1-16 by deletion of one or more terminal amino acids at the N-terminus or at the C-terminus of an individual SEQ ID NO. Variant or fragments preferably have a length of at least 8 amino acids or up to 10%, up to 20%, up to 30%, up to 40%, up to 50%, up to 60%, up to 70%, up to 80%, up to 90%, or up to 99% of its respective SEQ ID NO.

The term “human immunodeficiency virus” or “HIV”, as used herein, refers to human immunodeficiency viruses generically and includes HIV type 1 (“HIV-1”), HIV type 2 (“HIV-2”) or other HIV viruses, including, for example, the HIV-1, HIV-2, emerging HIV and other HIV subtypes and HIV variants, such as widely dispersed or geographically isolated variants and simian immunodeficiency virus (“SIV”). For example, an ancestral viral gene sequence can be determined for the env and gag genes of HIV-1, such as for HIV-1 subtypes A, B, C, D, E, F, G, H, J, and K, and intersubtype recombinants such as AG, AGI, and for groups M, N, O or for HIV-2 viruses or HIV-2 subtypes A or B. HIV-1, HIV-2 and SIV include, but are not limited to, extracellular virus particles and the forms of the viruses associated with their respective infected cells.

The term “operably linked”, as used herein, is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). See Auer H, Nature Biotechnol. 2006; 24: 41-43.

The term “peptide tag” or “tag”, as use herein, refers to a peptide or amino acid sequence, which can be used in the isolation or purification of said immunogen. Thus, said tag is capable of binding to one or more ligands, for example, one or more ligands of an affinity matrix such as a chromatography support or bead with high affinity. Illustrative, non-limitative, examples of tags useful for isolating or purifying a protein include Arg-tag, FLAG-tag, His-tag, or Strep-tag; an epitope capable of being recognized by an antibody, such as c-myc-tag (recognized by an anti-c-myc antibody), SBP-tag, S-tag, calmodulin binding peptide, cellulose binding domain, chitin binding domain, glutathione S-transferase-tag, maltose binding protein, NusA, TrxA, DsbA or Avi-tag; an amino acid sequence, such as AHGHRP (SEQ ID NO:53), PIHDHDHPHLVIHS (SEQ ID NO:54), or GMTCXXC (SEQ ID NO:55); or β-galactosidase. Terpe et al., Appl. Microbiol. Biotechnol. 2003; 60:523-525.

The term “secretion signal peptide” refers to a highly hydrophobic amino acid sequence (e.g., preferably 15 to 60 amino acids long) of proteins that must cross through membranes to arrive at their functioning cellular location. By binding to signal recognition particles, these sequences direct nascent protein-ribosome complexes to a membrane where the protein is inserted during translation. Signal peptides direct translational uptake of the protein by various membranes (e.g., endoplasmic reticulum, mitochondria, chloroplast, peroxisome). Leader signal sequences on non-membrane proteins are ultimately removed by specific peptidases. Some signal peptides used include MCP-3 chemokine, for promoting secretion and attraction of antigen presenting cells; a catenin (CATE)-derived peptide for increased proteasomal degradation; and the lysosomal associated protein, LAMP1 for targeting the MHC II compartment. Rosati et al., Proc. Natl. Acad. Sci. USA 2009; 106:15831-15836.

The expression “sequential administration”, as used herein, means that the administration is not simultaneous, but a first administration is performed, followed by one or more successive administrations.

The terms “prevent,” “preventing,” and “prevention”, as used herein, refer to inhibiting the inception or decreasing the occurrence of a disease in an animal. Prevention may be complete (e.g., the total absence of pathological cells in a subject). The prevention may also be partial, such that for example the occurrence of pathological cells in a subject is less than that which would have occurred without the present invention. Prevention also refers to reduced susceptibility to a clinical condition.

The term “treat” or “treatment”, as used herein, refers to the administration of an immunogenic composition of the invention or of a medicament containing it to control the progression of the disease before or after clinical signs have appeared. Control of the disease progression is understood to mean the beneficial or desired clinical results that include, but are not limited to, reduction of the symptoms, reduction of the duration of the disease, stabilization of pathological states (specifically to avoid additional deterioration), delaying the progression of the disease, improving the pathological state and remission (both partial and total). The control of progression of the disease also involves an extension of survival, compared with the expected survival if treatment was not applied.

The term “vaccine”, as used herein, refers to a substance or composition that establishes or improves immunity to a particular disease in a subject by inducing an adaptive immune response including an immunological memory. A vaccine typically contains an agent that resembles a disease-causing microorganism or a part thereof (e.g., a polypeptide). Vaccines can be prophylactic or therapeutic.

The term “vector”, as used herein, refers either a nucleic acid molecule or viral vector “comprising”, “containing” or “encoding”, as used herein, an immunogenic polypeptide described herein (e.g., the HTI immunogen). For example, a vector includes, but is not limited to, a nucleic acid vector (e.g., a nucleic acid molecule, linear or circular, operably linked to additional segments that provide for its autonomous replication in a host cell of interest or according to the expression cassette of interest). A vector also includes, but is not limited to, a viral vector “comprising”, “containing” or “encoding”, as used herein, an immunogenic polypeptide or nucleic acid molecule encoding an immunogenic polypeptide.

As used in the present disclosure and claims, the singular forms “a”, “an”, and “the” include plural forms unless the context clearly dictates otherwise.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Methods of Treating or Preventing an HIV Infection or a Disease Associated with an HIV Infection

In general terms, the present invention is directed to a method of treating or preventing an HIV infection or a disease associated with an HIV infection in a subject in need thereof, comprising administering the HTI immunogen of the invention to the subject in a priming step, followed by administering the HTI immunogen of the invention to the subject in a boosting step.

HTI Immunogens

The methods of the present invention relate to administration of HIV immunogens. International Pub. No. WO 2013/110818 and U.S. Pat. No. 9,988,425 (each of which is incorporated herein by reference in its entirety) describe immunogens for HIV vaccination (termed herein “HTI immunogens,” “HTI” or “immunogenic polypeptide(s)”). Sixteen regions in the Gag, Pol, Vif, and Nef proteins of the HIV-1 virus were relatively conserved and were targeted by HIV patients having a reduced viral load of <5000 copies of HIV-1 RNA per mL. Hancock et al., PLOS Pathogens 2015; 11(2): e1004658; Mothe et al., J. Translational Med. 2015; 13:60. These regions of HIV proteins formed the basis of an immunogen for therapeutic vaccination of HIV. The following table summarizes the regions of HIV-1 targeted by the immunogens:

TABLE 1

| HIV-1 protein | Position (HXB2) | SEQ ID NO |
|---|---|---|
| p17 | 17-94 | 1 |
| p24 | 30-43 | 2 |
| p24 | 61-71 | 3 |
| p24 | 91-150 | 4 |
| p24 | 164-177 | 5 |
| p24 | 217-231 | 6 |
| p2p7p1p6 | 63-89 | 7 |
| protease | 45-99 | 8 |
| reverse transcriptase | 34-50 | 9 |
| reverse transcriptase | 210-264 | 10 |
| reverse transcriptase | 309-342 | 11 |
| integrase | 210-243 | 12 |
| integrase | 266-282 | 13 |
| Vif | 25-50 | 14 |
| Vif | 166-184 | 15 |
| Nef | 56-68 | 16 |

The HIV numbering is as described in Korber et al., Human Retroviruses and AIDS 1998. Theoretical Biology and Biophysics Group, Los Alamos National Laboratory, Los Alamos, N.M., pp. III-102-111.

In some embodiments, the HTI immunogen can be administered through a heterologous prime-boost vaccination that includes different components and vectors, which can be selected from nucleic acids (for example, DNA and RNA vectors), viral vectors (for example, poxvirus, adenovirus, lentivirus, arenavirus and others), bacterial vectors, polypeptides, or antibodies. The aim of the sequential administration of the therapeutic vaccines is to achieve a so-called “functional cure”, in which HIV-infected participants could control viral replication in the absence of anti-retroviral treatment.

In some embodiments, the methods of the present invention comprise administration of a vector (e.g., viral or nucleic acid) encoding an immunogenic polypeptide (e.g., the HTI immunogen), wherein the immunogenic polypeptide comprises:
i. a sequence having at least 90%, 91%, 92%, 93%, 94%, 95%, 96%, 97%, 98%, or 99% sequence identity to SEQ ID NO:1;
ii. a sequence having at least 90%, 91%, 92%, 93%, 94%, 95%, 96%, 97%, 98%, or 99% sequence identity to SEQ ID NO:2;
iii. a sequence having at least 90%, 91%, 92%, 93%, 94%, 95%, 96%, 97%, 98%, or 99% sequence identity to SEQ ID NO:3;
iv. a sequence having at least 90%, 91%, 92%, 93%, 94%, 95%, 96%, 97%, 98%, or 99% sequence identity to SEQ ID NO:4;
v. a sequence having at least 90%, 91%, 92%, 93%, 94%, 95%, 96%, 97%, 98%, or 99% sequence identity to SEQ ID NO:5;
vi. a sequence having at least 90%, 91%, 92%, 93%, 94%, 95%, 96%, 97%, 98%, or 99% sequence identity to SEQ ID NO:6;
vii. a sequence having at least 90%, 91%, 92%, 93%, 94%, 95%, 96%, 97%, 98%, or 99% sequence identity to SEQ ID NO:7;
viii. a sequence having at least 90%, 91%, 92%, 93%, 94%, 95%, 96%, 97%, 98%, or 99% sequence identity to SEQ ID NO:8;
ix. a sequence having at least 90%, 91%, 92%, 93%, 94%, 95%, 96%, 97%, 98%, or 99% sequence identity to SEQ ID NO:9;
x. a sequence having at least 90%, 91%, 92%, 93%, 94%, 95%, 96%, 97%, 98%, or 99% sequence identity to SEQ ID NO:10;
xi. a sequence having at least 90%, 91%, 92%, 93%, 94%, 95%, 96%, 97%, 98%, or 99% sequence identity to SEQ ID NO:11;
xii. a sequence having at least 90%, 91%, 92%, 93%, 94%, 95%, 96%, 97%, 98%, or 99% sequence identity to SEQ ID NO:12;
xiii. a sequence having at least 90%, 91%, 92%, 93%, 94%, 95%, 96%, 97%, 98%, or 99% sequence identity to SEQ ID NO:13;
xiv. a sequence having at least 90%, 91%, 92%, 93%, 94%, 95%, 96%, 97%, 98%, or 99% sequence identity to SEQ ID NO:14;
xv. a sequence having at least 90%, 91%, 92%, 93%, 94%, 95%, 96%, 97%, 98%, or 99% sequence identity to SEQ ID NO:15; and
xvi. a sequence having at least 90%, 91%, 92%, 93%, 94%, 95%, 96%, 97%, 98%, or 99% sequence identity to SEQ ID NO:16. In some embodiments, at least two of the sequences (i)-(xvi) are joined by a single, dual, or triple alanine amino acid linker, wherein the linker results in the formation of an AAA sequence in the junction region between adjoining sequences, and/or wherein the sequence of each of (i) to (xvi) is 11-85, e.g., from 11 to 82, from 11 to 80, or from 11 to 78, amino acids in length.

In some embodiments, the immunogenic polypeptide comprises a sequence having amino acid sequences with no more than 1, 2, or 3 substitutions in any one of SEQ ID NOs: 1-16. In some embodiments, the immunogenic polypeptide comprises a sequence having amino acid sequences according to SEQ ID NOs: 1-16.

In some embodiments, the immunogenic polypeptide comprises an amino acid sequence having at least 90%, 91%, 92%, 93%, 94%, 95%, 96%, 97%, 98%, or 99% sequence identity to SEQ ID NO:17. In some embodiments, the immunogenic polypeptide comprises an amino acid sequence according to SEQ ID NO:17.

In some embodiments, the immunogenic polypeptide is encoded by any suitable nucleic acid sequence. In some embodiments, the immunogenic polypeptide is encoded by a nucleic acid sequence having at least 90%, 91%, 92%, 93%, 94%, 95%, 96%, 97%, 98%, or 99% sequence identity to SEQ ID NO:100 or 101. In some embodiments, the immunogenic polypeptide is encoded by a nucleic acid sequence of SEQ ID NO:100 or 101. In some embodiments, the nucleic acid encodes an immunogenic polypeptide comprising SEQ ID NO:99. In some embodiments, the nucleic acid is contained in a viral vector (e.g., a MVA or ChAd vector) or a nucleic acid vector.

In other embodiments, the immunogenic polypeptide comprises SEQ ID NOs:1-16. In other embodiments, the immunogenic polypeptide comprises the sequence of SEQ ID NOs:1-16 or a variant or fragment thereof. In some embodiments, the variant has a length of at least 8 amino acids, and does not comprise any sequence stretches derived from the HIV genome of a length of 8 or more amino acids other than an amino acid sequence according to any of SEQ ID NOs:1-16 or the variant thereof. In other embodiments, the variant is equivalent to its related sequence and derives from a different HIV strain or is an artificial HIV sequence. Equivalent in this respect means different in one or more amino acid residues, but corresponding to the same sequence (e.g., determined by the position in the genome or sequence similarity). In other words, in one embodiment, the variant is a “naturally occurring variant”, which refers to nucleic acid sequences derived from an HIV genome of a presently or formerly circulating virus and can be identified from existing databases (e.g., GenBank and Los Alamos sequence databases). The sequence of circulating viruses can also be determined by molecular biology methodologies. See Brown T, “Gene Cloning” (Chapman & Hall, London, G B, 1995); Watson et al., “Recombinant DNA”, 2nd Ed. (Scientific American Books, New York, N.Y., US, 1992); Sambrook et al., “Molecular Cloning. A Laboratory Manual” (Cold Spring Harbor Laboratory Press, Cold Spring Harbor, N.Y., US, 1989). In some embodiments, a variant of any of SEQ ID NOs:1-16 has an amino acid sequence identity of at least 70%, at least 80%, at least 90%, at least 95%, at least 98%, or at least 99% to its corresponding (i.e., SEQ ID NOs:1-16). Examples of algorithms suitable for determining percent sequence identity and sequence similarity are BLAST and BLAST 2.0 algorithms. Altschul et al., Nuc. Acids Res. 1977; 25:3389-3402 and Altschul et al., J. Mol. Biol. 1990; 215:403-410. The BLAST and BLAST 2.0 programs can be used to determine percent sequence identity for the nucleic acids and proteins of the invention. Software for performing BLAST analyses is publicly available through the National Center for Biotechnology Information. See http://blast.ncbi.nlm.nih.gov/blast.cgi, January 2012.

In some embodiments, the immunogenic polypeptide comprises at least two, at least three, or at least four sequences selected from SEQ ID NOs:1-16 or variants thereof, wherein when the immunogen comprises only two, three, or four sequences selected from SEQ ID NOs:1-16, then not all of these sequences are selected from the group consisting of SEQ ID NOs:3, 5, 6 and 16. In another embodiment, said immunogen has an amino acid sequence comprising at least two, at least three, at least four, at least five, at least six, at least seven, at least eight, at least nine, or at least ten sequences selected from SEQ ID NOs:1-16 or variants thereof, wherein when the immunogen comprises only two, three, four, five, six, seven, eight, nine or ten sequences selected from the group consisting of SEQ ID NOs:1-16, then not all of these sequences are selected from the group consisting of SEQ ID NOs:1-16.

In another embodiment, the variant or fragment has a length of 8 to 40 amino acids, for example, 11 to 27 amino acids. In some embodiments, the variant or fragment does not comprise an amino acid linker adjoining any of SEQ ID NOs:1-16. In some embodiments, the C-terminal amino acid of said variant or fragment is neither G, P, E, D, Q, N, T, S, nor C.

In some embodiments, the variant or fragment is combined with or fused to a heat shock protein, for example, Hsp10, Hsp20, Hsp30, Hsp40, Hsp60, Hsp70, Hsp90, gp96, or Hsp100.

In some embodiments, the variant or fragment is selected from SEQ ID NOs:17-45.

In some embodiments, at least two sequences of the immunogenic polypeptide are adjoined by an amino acid linker. In some embodiments, the linker has the amino acid sequence A, AA or AAA. In some embodiments, the C-terminal residue of the sequence located N-terminally with respect to the linker or the N-terminal residue of the sequence located C-terminally is an alanine residue, the linker can be shortened so that an AAA sequence is formed in the junction region between adjoining sequences. Thus, in some embodiments, if the C-terminal residue of the sequence located N-terminally with respect to the linker is an alanine or if the N-terminal residue of the sequence located C-terminally with respect to the linker is alanine, the linker has the sequence AA. In another embodiment, if the C-terminal residue of the sequence located N-terminally with respect to the linker and the N-terminal residue of the sequence located C-terminally with respect to the linker are both alanine, then the linker has the sequence A.

In another embodiment, the immunogenic polypeptide further comprises a secretion signal peptide at the N-terminus. In some embodiments, the signal peptide enhances secretion of the immunogen from cells expressing the immunogen. In some embodiments, the signal peptide is derived from GMCSF (granulocyte macrophage colony-stimulating factor), for example, followed by a valine to increase stability. The sequence of the GMCSF signal peptide is, for example, MWLQSLLLLGTVACSIS (SEQ ID NO:46) or MWLQSLLLLGTVACSISV (SEQ ID NO:47).

In another embodiment, the immunogenic polypeptide further comprises a peptide tag. In some embodiments, the peptide tag is located at the N-terminus between the signal peptide and the immunogenic polypeptide or at the C-terminus before the stop codon.

In some embodiments, the peptide tag is a FLAG peptide. The FLAG system utilizes a short, hydrophilic 8-amino acid peptide, which is fused to the recombinant protein of interest. The FLAG peptide includes the binding site for several highly specific ANTI-FLAG monoclonal antibodies (M1, M2, M5; Sigma-Aldrich Corp., Saint Louis, Mo., US), which can be used to assess expression of the protein of interest on material from transfected cells. Because of the small size of the FLAG peptide tag, it does not shield other epitopes, domains, or alter the function, secretion, or transport of the fusion protein generally. In some embodiments, the FLAG peptide has the sequence DYKDDDDKL (SEQ ID NO:48). In some embodiments, the peptide tag is only for expression analysis and/or purification of the immunogen and it is removed before using it to elicit an immune response.

In some embodiments, the sequence of the immunogenic polypeptide comprises at least one antiretroviral resistance mutation site.

In other embodiments, a nucleic acid encoding an immunogenic polypeptide described herein is also contemplated for the methods of the present invention. In some embodiments, the nucleic acid is a polynucleotide, single-stranded or double-stranded polymers of nucleotide monomers, including, but not limited to, 2′-deoxyribonucleotides (DNA) and ribonucleotides (RNA) linked by internucleotide phosphodiester bond linkages. In some embodiments, the nucleic acid comprises a promoter sequence, a 3′-UTR and/or a selection marker.

In one embodiment, said nucleic acid is codon optimized. In some embodiments, the nucleic acid is codon optimized for expression in humans. Codon-optimized nucleic acids for use according to the present invention can be prepared by replacing the codons of the nucleic acid encoding the immunogen by “humanized” codons (i.e., the codons are those that appear frequently in highly expressed human genes). André et al., J. Virol. 1998; 72:1497-1503. In one embodiment, the codon-optimized nucleic acid has the sequence according to SEQ ID NO:50.

Vectors

In some embodiments of the methods of the present invention, the HTI immunogen is administered via a vector. In some embodiments, the vector is an expression vector. In some embodiments, the vector is a DNA vector or a viral vector. Examples of vectors that can be used in the present invention include, but are not limited to, prokaryotic vectors, such as pUC18, pUC19, and Bluescript plasmids and derivatives thereof, like the mp18, mp19, pBR322, pMB9, ColEl, pCR1 and RP4 plasmids; phages and shuttle vectors, such as pSA3 and pAT28 vectors; expression vectors in yeasts, such as 2-micron plasmid type vectors; integration plasmids; YEP vectors; centromeric plasmids and analogues; expression vectors in insect cells, such as the vectors of the pAC series and of the pVL series; expression vectors in plants, such as vectors of the pIBI, pEarleyGate, pAVA, pCAMBIA, pGSA, pGWB, pMDC, pMY, pORE series and analogues; and expression vectors in superior eukaryotic cells based on viral vectors (e.g., modified vaccinia Ankara (MVA), adenoviruses (e.g., chimpanzee adenovirus (ChAd)), viruses associated to adenoviruses, retroviruses and lentiviruses) as well as non-viral vectors, such as the pSilencer 4.1-CMV (Ambion®, Life Technologies Corp., Carlsbad, Calif., US), pcDNA3, pcDNA3.1/hyg pHCMV/Zeo, pCR3.1, pEF1/His, pIND/GS, pRc/HCMV2, pSV40/Zeo2, pTRACER-HCMV, pUB6N5-His, pVAX1, pZeoSV2, pCI, pSVL, pKSV-10, pBPV-1, pML2d and pTDT1 vectors.

In some embodiments, the vector comprises a promoter and polyadenylation site. In some embodiments, the vector comprises a mammalian promoter and a polyadenylation site. In some embodiments, the promoter is the human cytomegalovirus (CMV) promoter. In some embodiments, the polyadenylation site is the bovine growth hormone (BGH) polyadenylation site. Vectors of the invention can be modified to optimize vector replication in bacteria and can further comprise a selection gene, for example, a gene coding a protein conferring resistance to an antibiotic. In some embodiments, the vector comprises a kanamycin resistance gene.

In some embodiments, the vector is a viral vector, for example, a virus containing a nucleic acid that codes for the HTI immunogen of the invention. In some embodiments, the virus has low toxicity and/or is genetically stable. In some embodiments, the viral vector is a retrovirus, for example, a poxvirus such as modified vaccinia Ankara (MVA), lentivirus, adenovirus such as chimpanzee adenovirus (ChAd), arenavirus or adeno-associated virus (AAV). In some embodiments, the MVA is a strain enhanced safety dueto with i) capability of reproductive replication in vitro in chicken embryo fibroblasts (CEF), but no capability of reproductive replication in a human cell line, as in the human keratinocyte cell line HaCaT, the human embryo kidney cell line 293, the human bone osteosarcoma cell line 143B, and the human cervix adenocarcinoma cell line HeLa; ii) failure to replicate in a mouse model that is incapable of producing mature B and T cells and as such is severely immune compromised and highly susceptible to a replicating virus; and iii) induction of at least the same level of specific immune response in vaccinia virus prime/vaccinia virus boost regimes when compared to DNA-prime/vaccinia virus boost regimes. In some embodiments, the MVA strain is MVA-BN. An exemplary MVA vector is described in Barouch et al. Cell; 2013, 155(3):531-539 (herein incorporated by reference in its entirety).

In some embodiments, the adenovirus is a simian adenovirus (SAds) or chimpanzee adenovirus (ChAd) (e.g., a replication deficient ChAd). Exemplary chimpanzee adenovirus vectors have been described, e.g., in U.S. Pat. No. 9,714,435 (incorporated by reference herein in its entirety).

In some embodiments, the methods of the present invention include administration of the HTI immunogen in a vector (e.g., a DNA vector or DNA.HTI described herein). DNA.HTI is a circular and double stranded deoxyribonucleic acid (DNA) plasmid vector of 5,676 base pairs derived from the pCMVkan expression vector backbone that contains the DNA encoding the 529 amino acid (aa) sequence for HTI. The HTI plasmid DNA contains the expression-optimized HTI open reading frame inserted into a pCMVkan vector comprising a plasmid backbone optimized for growth in bacteria, the human cytomegalovirus (CMV) promoter without any introns, the HTI gene, the bovine growth hormone (BGH) polyadenylation site, and the kanamycin resistance gene.

In other embodiments, the methods of the present invention include administration of the HTI immunogen in a MVA vector (e.g., MVA.HTI described herein). MVA.HTI is a live, attenuated recombinant vaccinia (pox) virus attenuated by serial passages in cultured chicken embryo fibroblasts (CEF) that contains six large deletions from the parental virus genome. A transgene coding for the insert HTI has been inserted within the MVA in order to induce an HIV-1 specific T cell immune response. The size of MVA.HTI after the insertion is estimated to be approximately 7,290 kbp.

In other embodiments, the methods of the present invention include administration of the HTI immunogen in a chimpanzee adenovirus vector (e.g., ChAdOx1.HTI is a replication-defective recombinant chimpanzee adenovirus (ChAd) vector based on a chimpanzee adenoviral isolate Y25. ChAdOx1.HTI is a replication-defective recombinant chimpanzee adenovirus (ChAd) vector based on a chimpanzee adenoviral isolate Y25 that encodes the HTI sequence. ChAdOx1.HTI was derived by sub-cloning the HTI antigen sequence into the generic ChAdOx1 BAC in order to induce HIV-1 specific T-cell immune response. The plasmid resulting from this sub-cloning (pC255; 40,483 kbp) was linearized and transfected into commercial HEX293A T-REx® cells to produce the vectored vaccine ChAdOx1.HTI.

Additional Dosing and Dosing Regimens

In some embodiments, the method of the present invention comprises (a) administering to the subject 1 to 10 (i.e., 1, 2, 3, 4, 5, 6, 7, 8, 9 or 10) administrations of a vector (e.g., DNA vector or viral vector) encoding the HTI immunogen, and (b) administering to the subject 1 to 10 (i.e., 1, 2, 3, 4, 5, 6, 7, 8, 9 or 10) administrations of a vector (e.g., DNA vector or viral vector) encoding the HTI immunogen. In some embodiments, the method comprises (a) administering to the subject 1 to 10 (i.e., 1, 2, 3, 4, 5, 6, 7, 8, 9 or 10) administrations of a DNA vector encoding the HTI immunogen, followed by 1 to 10 (i.e., 1, 2, 3, 4, 5, 6, 7, 8, 9 or 10) administrations of a first viral vector encoding the HTI immunogen; and (b) administering to the subject 1 to 10 (i.e., 1, 2, 3, 4, 5, 6, 7, 8, 9 or 10) administrations of a second viral vector encoding the HTI immunogen.

In some embodiments, the method comprises (a) administering to the subject 1 to 4 administrations of a vector (e.g., a DNA vector or viral vector) encoding the HTI immunogen, and (b) administering to the subject 1 to 4 administrations of a vector (e.g., DNA vector or viral vector) encoding the HTI immunogen. In some embodiments, the method comprises (a) administering to the subject 1 to 4 administrations of a DNA vector encoding the HTI immunogen, followed by 1 to 4 administrations of a first viral vector encoding the HTI immunogen; and (b) administering to the subject 1 to 4 administrations of a second viral vector encoding the HTI immunogen.

In some embodiments, the method comprises (a) administering to the subject 3 administrations of a DNA vector encoding the HTI immunogen, followed by 2 administrations of a first viral vector encoding the HTI immunogen. In some embodiments, the method comprises (b) administering to the subject 3 administrations of the first viral vector encoding the HTI immunogen. In some embodiments, the method comprises (b) administering to the subject 2 administrations of a second viral vector encoding the immunogenic polypeptide, followed by 1 administration of a first viral vector encoding the immunogenic polypeptide. In some embodiments, the method comprises (a) administering to the subject 3 administrations of a DNA vector encoding the HTI immunogen, followed by 2 administrations of a first viral vector encoding the HTI immunogen; and (b) administering to the subject 3 administrations of a second viral vector encoding the HTI immunogen. In some embodiments, the method comprises (a) administering to the subject 3 administrations of a DNA vector encoding the immunogenic polypeptide, followed by 2 administrations of an MVA vector encoding the immunogenic polypeptide; and (b) administering to the subject 2 administrations of a ChAd vector encoding the immunogenic polypeptide, followed by 1 administration of a MVA vector encoding the immunogenic polypeptide.

In some embodiments, the method comprises (a) administering to the subject (i) 3 administrations of a DNA vector encoding an immunogenic polypeptide (e.g., HTI immunogen), each separated by a period of about 4 weeks; (ii) 1 administration of a first viral vector encoding the immunogenic polypeptide about 4 weeks after (a)(i); and (iii) 1 administration of a first viral vector encoding the immunogenic polypeptide about 8 weeks after (a)(ii); and (b) administering to the subject (i) 2 administrations a second viral vector encoding the immunogenic polypeptide, each separated by a period of about 12 weeks; and (ii) 1 administration of the first viral vector encoding the immunogenic polypeptide about 12 weeks after (b)(i); wherein the administering of (b) is separated from the administering of (a) by a period of about 24 weeks. In some embodiments of such a method, the administrations of (a)(i) are at a dose of about 4 mg, the administration of (a)(ii) is at a dose of about 2×10⁸ pfu, the administration of (a)(iii) is at a dose of about 2×10⁸ pfu, the administrations of (b)(i) are at a dose of about 5×10¹⁰ viral particles, and/or the administration of (b)(ii) is at a dose of about 2×10⁸ pfu. In other embodiments, the DNA vector of (a)(i) comprises a human cytomegalovirus (CMV) promoter and/or a bovine growth hormone (BGH) polyadenylation site. In other embodiments, the first viral vector is an MVA vector. In other embodiments, the second viral vector is a ChAd vector.

In some embodiments, the method comprises (a) administering to the subject 1 to 5 administrations of a first viral vector encoding an immunogenic polypeptide. In some embodiments, the method comprises (b) administering to the subject 1 to 5 administrations of a second viral vector encoding the immunogenic polypeptide. In some embodiments, the method comprises (a) administering to the subject 1 to 5 administrations of a first viral vector encoding the immunogenic polypeptide; and (b) administering to the subject 1 to 5 administrations of a second viral vector encoding the immunogenic polypeptide. In some embodiments, the method comprises (a) administering to the subject 2 administrations of a ChAd vector encoding the immunogenic polypeptide. In some embodiments, the method comprises (b) administering to the subject 2 administrations of an MVA vector encoding the immunogenic polypeptide. In some embodiments, the method comprises (a) administering to the subject 2 administrations of a ChAd vector encoding the immunogenic polypeptide; and (b) administering to the subject 2 administrations of an MVA vector encoding the immunogenic polypeptide.

In other embodiments, the method comprises (a) administering to the subject 2 administrations of a first viral vector encoding the immunogenic polypeptide, each separated by a period of about 12 weeks; and (b) administering to the subject 2 administrations of a second viral vector encoding the immunogenic polypeptide, each separated by a period of about 12 weeks; and wherein the administering of (b) is separated from the administering of (a) by a period of about 12 weeks. In some embodiments, the administrations of (a) are at a dose of about 5×10¹⁰ viral particles, and/or the administrations of (b) are at a dose of about 2×10⁸ pfu. In some embodiments, the first viral vector is a ChAd vector. In some embodiments, the second viral vector is an MVA vector.

Immunogenic polypeptides and polynucleotides and vectors encoding the same of the invention can be administered in a variety of manners, for example, via the mucosa, such as oral and nasal, pulmonary, intramuscular, subcutaneous or intradermal routes.

Immunogenic polypeptides and polynucleotides and vectors encoding the same of the invention can also be administered in a pharmaceutical composition comprising a pharmaceutically acceptable carrier (also referred to herein as a vaccine or vaccine formulation). Examples of a pharmaceutically acceptable carrier include, but are not limited to, a solid, semisolid or liquid filler, diluent, encapsulating material or formulation auxiliary of any conventional type. Other suitable pharmaceutically acceptable carriers include, but are not limited to, water, dextrose, glycerol, saline, ethanol, and combinations thereof. In some embodiments, a pharmaceutically acceptable carrier can contain additional agents such as wetting or emulsifying agents, pH buffering agents, or adjuvants that enhance the effectiveness of the formulation.

In addition, aqueous compositions, such as those used to prepare HIV vaccine formulations, may be prepared in sterile form, and when intended for delivery by other than oral administration generally may be isotonic. All compositions may optionally contain excipients such as those set forth in the Rowe et al, Handbook of Pharmaceutical Excipients, 6^{th} edition, American Pharmacists Association, 2009. Excipients can include ascorbic acid and other antioxidants, chelating agents such as EDTA, carbohydrates such as dextrin, hydroxyalkylcellulose, hydroxyalkylmethylcellulose, stearic acid and the like.

In some embodiments, a pharmaceutical composition comprises 0.5 mL Tris buffer (10 mM Tris HCl, pH 7.7, 140 mM NaCl). In some embodiments, the pharmaceutical composition comprises 2×10⁸ plaque forming units (PFU) of a viral vector encoding the HTI immunogen in 0.5 mL Tris buffer (10 mM Tris HCl, pH 7.7, 140 mM NaCl). In some embodiments, the pharmaceutical composition comprises 2×10⁸ plaque forming units (PFU) of an MVA vector encoding the HTI immunogen in 0.5 mL Tris buffer (10 mM Tris HCl, pH 7.7, 140 mM NaCl). In some embodiments, the pharmaceutical composition comprises 2×10⁸ plaque forming units (PFU) of an MVA vector comprising a nucleic acid encoding an immunogenic polypeptide having an amino acid sequence of SEQ ID NO:99 in 0.5 mL Tris buffer (10 mM Tris HCl, pH 7.7, 140 mM NaCl). In some embodiments, the pharmaceutical composition comprises 2×10⁸ PFU of an MVA vector comprising a nucleic acid comprising the sequence of SEQ ID NO:100 or 101 in 0.5 mL Tris buffer (10 mM Tris HCl, pH 7.7, 140 mM NaCl).

It should be understood that description herein related to the administration of an immunogenic polypeptide or nucleic acid encoding an immunogenic polypeptide also applies to administration of a pharmaceutical composition or vaccine containing the same.

The amount of the virus within a pharmaceutical composition can be measured by any means known in the art. The amount may be determined by bulk measurement of the number of viral particles (vp) within an amount of aqueous composition, e.g., by flow cytometry. Alternatively, the amount may be determined by the activity of the virus within the composition, e.g., by plaque assay. Plaque-based assays can be used to determine virus concentration in terms of infectious dose. Viral plaque assays determine the number of plaque forming units (pfu) in a virus sample, which can be used as a measure of virus quantity. Kaufmann et al. 2002; Methods in Microbiology Vol. 32: Immunology of Infection. Academic Press. ISBN 0-12-521532-0.

In some embodiments, a DNA vector encoding an immunogenic polypeptide of the present invention is administered at a dose of from between about 0.1 mg and about 20 mg, for example, from about 0.1 mg to about 15 mg, from about 0.1 mg to about 10 mg, from about 0.1 mg to about 5 mg, from about 0.1 mg to about 1 mg, from about 1 mg to about 20 mg, from about 1 mg to about 15 mg, from about 1 mg to about 10 mg, from about 1 mg to about 5 mg, from about 5 mg to about 20 mg, from about 5 mg to about 10 mg, from about 10 mg to about 20 mg, from about 10 mg to about 15 mg, or from about 15 mg to about 20 mg. In some embodiments, a DNA vector encoding an immunogenic polypeptide of the present invention is administered at a dose of from between about 0.5 mg and about 10 mg. In some embodiments, a DNA vector encoding an immunogenic polypeptide of the present invention is administered at a dose of from about 1 mg to about 8 mg. In some embodiments, a DNA vector is administered at a dose of about 0.1 mg, about 0.5 mg, about 1 mg, about 2 mg, about 3 mg, about 4 mg, about 5 mg, about 10 mg, about 15 mg or about 20 mg.

In some embodiments, a viral vector (e.g., MVA or ChAd vector) encoding an immunogenic polypeptide of the present invention is administered at a dose of from about 1×10⁷ plaque forming units (pfu) to about 1×10⁹ pfu, for example, from about 5×10⁷ pfu to about 1×10⁹ pfu, from about 1×10⁸ pfu to about 1×10⁹ pfu, from about 5×10⁸ pfu to about 1×10⁹ pfu. In some embodiments, a viral vector encoding an immunogenic polypeptide of the present invention is administered at a dose of from about 5×10⁷ pfu to about 5×10⁸ pfu. In some embodiments, a viral vector encoding an immunogenic polypeptide of the present invention is administered at a dose of about 2.5×10⁸ pfu. In some embodiments, a viral vector encoding an immunogenic polypeptide of the present invention is administered at a dose of about 1×10⁷ pfu, about 1×10⁸ pfu, about 1×10⁹ pfu, about 5×10⁷ pfu or about 5×10⁸ pfu.

In some embodiments, a viral vector (e.g., MVA or ChAd vector) encoding an immunogenic polypeptide of the present invention is administered at a dose of from about 1×10⁹ viral particles and 5×10¹¹ viral particles, for example, from about 5×10⁹ pfu to about 5×10¹¹ pfu, from about 1×10¹⁰ pfu to about 5×10¹¹ pfu, from about 5×10¹⁰ pfu to about 5×10¹¹ pfu, or from about 1×10¹¹ pfu to about 5×10¹¹ pfu. In some embodiments, a viral vector encoding an immunogenic polypeptide of the present invention is administered at a dose of from about 1×10¹⁰ to about 1×10¹¹ viral particles, for example, from about 5×10¹⁰ pfu to about 1×10¹¹ pfu. In some embodiments, a viral vector encoding an immunogenic polypeptide of the present invention is administered at a dose of from about 5×10¹⁰ viral particles.

The amount of immunogenic compound (e.g., HTI immunogen) delivered can vary, depending upon the intended use (preventive or therapeutic vaccination), and age and weight of the subject to be immunized, the vaccination protocol adopted (i.e., single administration versus repeated doses), the route of administration and the potency and dose of the adjuvant compound chosen. The amount can be ascertained by standard studies involving observation of appropriate immune responses in vaccinated subjects. In some embodiments, following an initial vaccination, composed itself by one or several doses, subjects can receive one or several booster immunization adequately spaced.

In some embodiments, an immunogenic compound or composition is administered on an one off basis, or can be administered repeatedly, for example, from about 1 and about 10 times, for example, from about 1 to about 9 times, from about 1 to about 8 times, from about 1 to about 7 times, from about 1 to about 6 times, from about 1 to about 5 times, from about 1 to about 4 times, from about 1 to about 3 times, from about 1 to about 2 times, from about 2 to about 9 times, from about 2 to about 8 times, from about 2 to about 7 times, from about 2 to about 6 times, from about 2 to about 5 times, from about 2 to about 4 times, from about 2 to about 3 times, from about 3 to about 9 times, from about 3 to about 8 times, from about 3 to about 7 times, from about 3 to about 6 times, from about 3 to about 5 times, from about 3 to about 4 times, from about 4 to about 9 times, from about 4 to about 8 times, from about 4 to about 7 times, from about 4 to about 6 times, or from about 4 to about 5 times.

In some embodiments, an immunogenic compound or composition is administered at different intervals between doses of the same component or doses of different component. In some embodiments, the interval between doses is from about 1 week to about 24 months, for example, from about 2 weeks to about 24 months, from about 3 weeks to about 24 months, from about 4 weeks to about 24 months, from about 2 weeks to about 56 weeks, from about 4 weeks and about 12 weeks.

In other embodiments, each administration of the methods of the present invention is separated by a period of from about 15 days to about 18 months. In some embodiments, each administration of the methods of the present invention is separated by a period of from about 1 week to about 24 months. In some embodiments, each administration of the methods of the present invention is separated by a period of from about 2 weeks to about 56 weeks. In some embodiments, each administration of the methods of the present invention is separated by a period of from about 4 weeks to about 12 weeks. In some embodiments of the methods of the present invention, the administering of step (a) of the methods of the present invention is separated from the administering of step (b) by a period of from about 2 months to about 24 months. In some embodiments of the methods of the present invention, the administering of step (a) is separated from the administering of step (b) by a period of from about 3 months to about 18 months.

In some embodiments, the vaccine components of the present invention can be grouped in a priming phase and a subsequent one or multiple boosting phases. In some embodiments, the priming phase and the boosting phase can be separated by from about 2 months to about 24 months, for example, from about 3 months to about 18 months. In some embodiments, the subject will receive the immunogen compound or composition of the invention as different vaccine components in a prime-boost regime. In some embodiments, such a regimen is followed by dosing at regular intervals of from about 1 months to about 12 months for a period up to the remainder of the subject's life.

In some embodiments, the immunogenic compounds or compositions of the invention are used in any sequence, each component be used one or several times, in any order, and with any interval between doses. For example, a sequence is DNA.HTI (D)+MVA.HTI (M)+ChAd.HTI (C) (vaccination sequence DMC), each dose separated 4 weeks apart. In some embodiments, the sequence comprises a priming phase of DDD (4 mg each) each dose administered at 4 weeks interval, followed by a M dose of 2×10⁸ pfu 4 weeks after the last D dose, followed by a second M dose of 2×10⁸ pfu 8 weeks after the last M dose, followed by a boosting phase at least 24 weeks apart comprising two C doses of 5×10¹⁰ viral particles each separated 12 weeks apart followed by a third M dose of 2×10⁸ pfu 12 weeks after the last C dose. Thus, the full sequence is: (1) Priming phase of DDDMM, followed 24 weeks after by a (2) boosting phase of CCM.

In some embodiments, the sequence comprises a priming phase of CC (5×10¹⁰ viral particles each), at week 0 and week 12, followed by a boosting phase of a first dose of M 12 weeks after the last C and a second dose of M 12 weeks after the first M (each dose of M of 2×10⁸ pfu).

HIV Infection or a Disease Associated with an HIV Infection and Other Methods

In some embodiments, the present invention is directed to a method of treating or preventing HIV infection or a disease associated with an HIV infection. In some embodiments, the HIV is HIV type 1 (HIV-1). In some embodiments, the HIV is HIV type 2 (HIV-2).

In some embodiments, the disease associated with an HIV infection is an acquired immune deficiency syndrome (AIDS), AIDS-related complex (ARC), or HIV opportunistic disease. In some embodiments, the HIV opportunistic disease is Burkitt's lymphoma, candidiasis in the bronchi, trachea, lungs, or esophagus, cervical cancer, coccidioidomycosis (disseminated or outside the lungs), cryptococcosis (outside the lungs), cryptosporidiosis (in the intestines lasting for more than 1 month), cytomegalovirus infection (outside the liver, spleen, or lymph nodes), cytomegalovirus retinitis (with loss of vision), HIV encephalopathy, herpes simplex lesions lasting for more than one month, herpes simplex m the bronchi, lung, or esophagus, histoplasmosis (disseminated or outside the lungs), immunoblastic lymphoma, invasive cervical carcinoma (cancer), isosporiasis in the intestines lasting for more than one month, Kaposi's sarcoma, lymphoma (primary in the brain), Mycobacterium avium complex (disseminated or outside the lungs), Mycobacterium kansasii (disseminated or outside the lungs), Mycobacterium tuberculosis (disseminated or outside the lungs), Pneumocystis carinii pneumonia, pneumonia (recurrent in 12 month period), progressive multifocal leukoencephalopathy (PML), salmonella septicemia (recurrent), toxoplasmosis (in the brain), wasting syndrome or any other disease resulting from an infection facilitated by a compromised immune system in an HIV-infected patient.

In some embodiments of the methods of the present invention, one or more of the following clinical effects are observed in non-HIV-infected subjects: avoiding the HIV infection in at least 30% of vaccinated individuals, or more desirably avoiding the HIV infection in more than 50% of vaccinated individuals. In some embodiments, the HIV is HIV-1.

In some embodiments of the methods of the present invention, one or more of the following clinical effects are observed in HIV-infected subjects: (1) a substantial reduction of the HIV-1 viral load in the subject's blood and tissues for a significant amount of time (non-progressor phenotype), typically under 2,000 copies of RNA per ml of plasma, or more desirably, under 50 copies of RNA per ml of plasma (2) a reduction or remission in AIDS-related clinical symptoms, and (3) a reduction in the conventional antiretroviral treatment, more desirably the complete interruption of the cART. A reduction or remission of AIDS-related clinical symptoms includes, but is not limited to, prolonging the asymptomatic phase of HIV infection; maintaining low viral loads in HIV infected patients whose virus levels have been lowered via anti-retroviral therapy (ART); increasing levels of CD4 T cells or lessening the decrease in CD4 T cells, both HIV-1 specific and non-specific, in drug naive patients and in patients treated with ART, increasing the breadth, magnitude, avidity and functionality of HIV specific CTL, increasing overall health or quality of life in an individual with AIDS; and prolonging life expectancy of an individual with AIDS. A clinician can compare the effect of immunization with the patient's condition prior to treatment, or with the expected condition of an untreated patient, to determine whether the treatment is effective in inhibiting AIDS.

In some embodiments, the methods of the present invention relate to generating a T-cell cellular response in a subject by administration of an immunogenic polypeptide described herein using a dosing regimen described herein.

In some embodiments, the methods of the present invention generate an effective cytotoxic T cell response. A cytotoxic T cell or cytotoxic T lymphocyte (CTL) assay can be used to monitor the cellular immune response following subgenomic immunization with a viral sequence against homologous and heterologous HIV strains. Burke et al., J. Inf. Dis. 1994; 170:1110-1119 and Tigges et al., J. Immunol, 1996; 156:3901-3910. Conventional assays utilized to detect T cell responses include, for instance, proliferation assays, lymphokine secretion assays, direct cytotoxicity assays and limiting dilution assays. For example, antigen-presenting cells that have been incubated with a peptide can be assayed for their ability to induce CTL responses in responder cell populations. Antigen-presenting cells can be cells such as peripheral blood mononuclear cells (PBMCs) or dendritic cells (DCs). Alternatively, mutant non-human mammalian cell lines that are deficient in their ability to load MHC class I molecules with intemally processed peptides and that have been transfected with the appropriate human MHC class I gene, can be used to test the capacity of a peptide of interest to induce in vitro primary CTL responses. PBMCs can be used as the responder cell source of CTL precursors. The appropriate antigen-presenting cells are incubated with the peptide after which the protein-loaded antigen-presenting cells are incubated with the responder cell population under optimized culture conditions. Positive CTL activation can be determined by assaying the culture for the presence of CTL that kill radiolabeled target cells, both specific peptide-pulsed targets as well as target cells expressming endogenously processed forms of the antigen from which the peptide sequence was derived. For example, the target cells can be radiolabeled with ⁵¹Cr and cytotoxic activity can be calculated from radioactivity released from the target cells. Another suitable method allows the direct quantification of antigen-specific T cells by staining with fluorescein-labeled HLA tetrameric complexes. Altman et al., Proc. Natl. Acad. Sci. USA 1993; 90:10330-10334 and Altman et al., Science 1996; 274:94-96. Other relatively recent technical developments include staining for intracellular lymphokines and interferon release assays or ELISpot assays.

In some embodiments of the methods of the present invention, the subject is a human subject.

Kits

In some embodiments, the present invention relates to a kit comprising immunogenic polypeptide of the invention, or nucleic acid or vector encoding the same, or a pharmaceutical composition comprising the same, and instructions for using the same in a method of present invention described herein. In some embodiments, the kit comprises a packaging, such as glass, plastic (e.g., polyethylene, polypropylene, polycarbonate), bottles, vials, paper, or sachets for the components. In some embodiments, the instructions are in the form of printed material or in the form of an electronic support which can store the instructions, for example, electronic storage media (e.g., magnetic disks, tapes), or optical media (e.g., CD-ROM, DVD). The media can additionally or alternatively contain internet websites providing such instructions.

All publications, patents and patent applications mentioned in this application are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

Embodiments of the present disclosure can be further defined by reference to the following non-limiting examples, which describe in detail preparation of some antibodies of the present disclosure and methods for using antibodies of the present disclosure. It will be apparent to those skilled in the art that many modifications, both to materials and methods, may be practiced without departing from the scope of the present disclosure.

### EXAMPLES

### Example 1

Construction of the ChAdOx1.HTI Vaccine

ChAdOx1.HTI is a replication-defective recombinant chimpanzee adenovirus (ChAd) vector based on a chimpanzee adenoviral isolate Y2546 that encodes the HTI sequence. ChAdOx1.HTI was derived by sub-cloning the HTI antigen sequence into the generic ChAdOx1 BAC (Oxford University). The plasmid resulting from this sub-cloning (pC255; 40,483 kbp) was linearized and transfected into commercial HEX293A T-REx® cells to produce the vectored vaccine ChAdOx1.HTI. ChAdOx1.HTI was formulated as a suspension for intramuscular (i.m.) injection. The buffer for injection contained 10 mM of L-Histidine, 35 mM of NaCl, 7.5% (w/v) of sucrose, 1 mM of MgCl₂, 0.1 mM of EDTA disodium, 0.1% (w/v) of Polysorbate-80, and 0.5% (v/v) of ethanol. pH was adjusted with HCl to 6.6. Vials were stored at −80° C.

### Example 2

Response of Single Vaccination to ChAdOx1.HTI in C57/b16 Mice

C57/b16 mice (female) were vaccinated with a single dose of ChAdOx1.HTI, according to the following schedule: Group 1: 1×10⁷ Vp; Group 2: 1×10⁸ Vp; Group 3: 1×10⁹ Vp; Group 4: 1×10¹⁰ Vp; and Group 5: Vehicle. Five C57Bl/6 mice per group were immunized with 100 μL volume (50 μL per leg) at different doses. Data showed a broad and strong immune response to HTI and a general dose-response increase in the number of SFCs according to the number of Vp administered. High-resolution analysis using 17 peptide pools showed that at least 11 epitopes of HTI were targeted by T cell responses.

### Example 3

Construction of the ChAdNous68-HIVACAT Vaccine

The vector ChAdNou68 hCMV tetO HTI BGHPolyA was created using transgene of ChAdox hCMV tetO HTI BGHPolyA. The virus was produced in suspension cell line M9.S and purified at P2 with CsCl2 purification, desalting and recovery of the purified adenovirus.

### Example 4

Comparison of the Immunogenicity of ChAdOx1.HTI with ChAdNous68.HTI in C57/b16 Mice

Immunogenicity after a single intramuscular vaccination with either vector was measured in seven weeks old C57/b16 female mice. To test different vaccine doses, a total of six groups of six mice each were immunized in both quadriceps, three groups receiving three doses of ChAdOx1.HTI and three groups receiving three doses of ChAdNou68.HTI. The tested doses were: 10⁷, 10⁸ and 10⁹ viral particles (VP)/animal respectively.

Three weeks after a single-dose vaccination, animals were sacrificed to aseptically recover spleens. Splenocytes were isolated mechanistically using a 40 μm filter (Corning), washed and resuspended in R10 media containing Roswell Park Memorial Institute (RPMI, Gibco), 2 mM of L-glutamine (Gibco), 100 U/ml penicillin (Gibco), 100 μg/ml streptomycin (Gibco) and 10% of fetal bovine sera (FBS, Gibco). The breadth and magnitude of T cell responses in the isolated splenocytes were measured using an INFy-ELISPOT assay (Mabtech). Briefly, 0.45 μm Hydrophobic High Protein Binding, Immobilon-P Membranes (MSIPS4W10, Millipore) were coated with anti-mouse IFNγ monoclonal antibody (mAb) AN18 and used to measure INFγ production incubating 400,000 splenocytes per well with 17 pools of 15mer partially overlapping peptides covering the entire HTI sequence. Each peptide pool contained a median of 8 overlapping peptides at individual peptide concentration of 14 ug/ml per peptide. After an 18 hours incubation period at 37 C, the cells were washed out and the INFγ bound to the capture antibody detected using the biotinylated anti-mouse IFN-γ mAb R4-6A2, alkaline phosphatase (ALP) labeled streptavidin and the alkaline phosphatase conjugate substrate Kit (BioRad). As negative control of the assay, R10 medium was used in triplicates and as positive control, 7 μg/ml of concanavalin A was added to positive control wells. Spot forming cells (SFC) were counted using the BioSpot automated spot counter (Immunospot, CTL). The cut-off for a positive response was defined as responses exceeding the highest of i) 5 spots/well, ii) 3 times the mean SFC in the negative control triplicates or iii) the mean SFC in the three negative controls plus 3 times the standard deviation of the SFC in the negative control wells. From the INFγ-ELISPOT assay, the breath (number of positive pools/animal) and magnitude (total SFC/10⁶ splenocytes) of the responses were calculated. Since the data didn't follow a normal distribution median and interquartile range (IQR) were used and significant differences in the breadth and magnitude of the HTI-specific responses between groups was tested by non-parametric Mann-Whitney t-test.

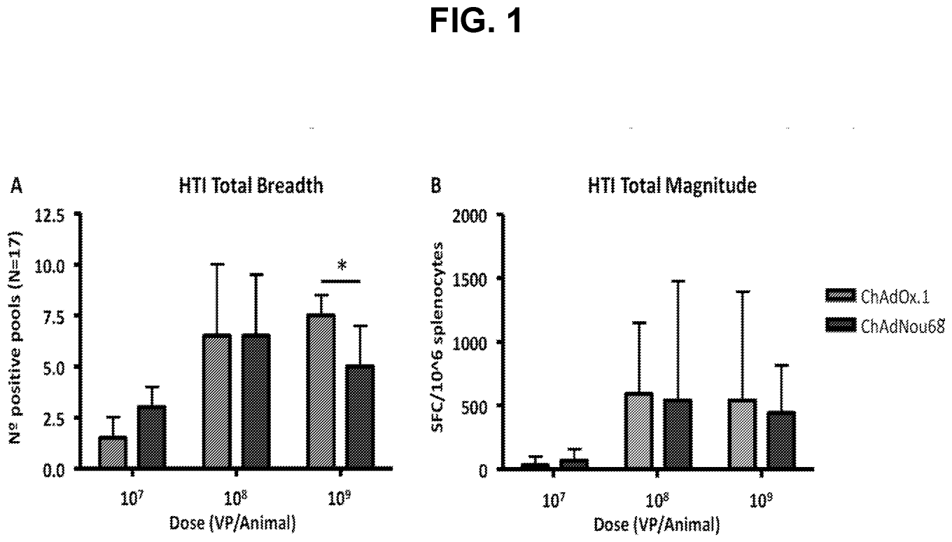

Comparing the responses obtained using the two different vectors, both vectors performed similarly at all doses, both in terms of total breadth (FIG. 1A) and magnitude (FIG. 1B). Only at the highest dose tested (10⁹ VP/animal), the Ox1 vector induced stronger and broader responses compared to ChAdNou68.HTI. The increase in the breath of the response, with a median of 7.5 positive pools for ChAdOx1.HTI compared to a median of 5 positive pools for ChAdNou68.HTI and reached statistical significance (Mann-Whitney p=0.0260) while the difference in the total magnitude (median of 540 SFC/10⁶ splenocytes for ChAdOx1.HTI compared and 440 SFC/106 splenocytes for ChAdNou68.HTI) at this dose was not statistically different (Mann-Whitney p=0.3095).

### Example 5

Immune Responses of Combinations of DNA.HTI, MVA.HTI and ChAdOx1.HTI in C57BL/6 Mice

Five or six days before treatment, C57BL/6 mice (males and females) were randomized according to body weight criteria in 8 groups (group 1a, 1b, 1c, 2a, 2b, 3a, 3b and 4) composed of 5 males and 5 females. Females and males of a same group were divided in two different cages. The DNA. HTI buffer was sterile phosphate buffered saline (PBS) without Ca2+ and Mg2+(OZYME, France). The MVA HTI buffer (Tris buffer) was prepared by diluting in water Tris (hydroxymethyl) aminomethane base (Sigma) at a concentration of 1.1 mg/ml and sodium chloride (Sigma) at a concentration of 8.18 mg/ml. pH was adjusted at 7.7±0.3. The ChAdOx1.HTI buffer contained 10 mM of L-Histidine, 35 mM of NaCl, 7.5% (w/v) of sucrose, 1 mM of MgCl₂, 0.1 mM of EDTA disodium, 0.1% (w/v) of Polysorbate-80, 0.5% (v/v) of ethanol. pH was adjusted with HCl to 6.6 and Endotoxin was tested <0.5 EU/ml. DNA.HTI (Batch No. PB125) was constructed as described in Int'l Pub. No. WO 2013/110818, and provided as 11 vials of 1.2 ml at a concentration of 4 mg/ml. The test substance was stored at −80° C. and diluted the day of the treatment at the appropriate concentration.

MVA.HTI was constructed as described in was constructed as described in Intl Pub. No. WO 2013/110818, and was supplied as vial at a concentration of 4×10⁸ PFU/ml. Before each administration a fresh solution was prepared daily. The MVA.HTI was diluted in the MVA.HTI vehicle in order to obtain a final concentration of 1×10⁶ PFU/ml which allowed an injection at 5×10⁴ PFU/50 μl (injection volume) for each site and therefore a total injection dose of 1×10⁵ PFU. The MVA.HTI was stored at −80° C. and diluted the day of the treatment at the appropriate concentration.

ChAdOx1.HTI was supplied as vial at a concentration of 1×10¹¹ vp/ml. Before each administration, a fresh solution was prepared daily. The ChAdOx1.HTI was diluted in the ChAdOx1.HTI vehicle (L-Histidine: 10 mM NaCl: 35 mM Sucrose: 7.5% (w/v) MgCl₂: 1 mM; EDTA disodium: 0.1 mM Tween 80 (Polysorbate-80): 0.1% (w/v) Ethanol 0.5%: (v/v) HCl: Adjusted to pH 6.6) in order to obtain a final concentration of 1×10⁹ vp/ml which allowed an injection at 5×10⁷vp/50 μl (injection volume) for each site and therefore a total injection dose of 1×10⁸ vp per mouse. The ChAdOx1.HTI was stored at −80° C. and diluted the day of the treatment at the appropriate concentration.

The test substances and the vehicle (50 μl/injection site, 2 injection sites) were administered by intramuscular injection (IM), in the two thighs of mice for each treatment using an insulin syringe according to the following groups:

Ten mice (5 females, 5 males) from group 1a (G1a) were treated by two intramuscular (IM) injections (one injection in each thigh of the mouse) of DNA.HTI at day one (D1), D8 and D15. Then, all mice were treated with MVA.HTI at D22 and D36.

Ten mice (5 females, 5 males) from group 1b (G1b) were treated by two IM injections (one injection in each thigh of the mouse) of DNA.HTI at D1, D8 and D15. Then, all mice were treated with MVA.HTI at D22 and D36.

Ten mice (5 females, 5 males) from group 1c (G1c) were treated by two IM injections (one injection in each thigh of the mouse) of DNA.HTI at D1, D8 and D15. Then, all mice were treated with MVA.HTI at D22 and D36. At D92 and D106, mice were treated with ChAdOx1.HTI and finally at D120, mice were treated with MVA.HTI.

Ten mice (5 females, 5 males) from group 2a (G2a) were treated by two IM injections (one injection in each thigh of the mouse) of vehicle DNA.HTI buffer at D1, D8 and D15, the vehicle MVA.HTI buffer at D22 and D36. Then, all mice were treated with ChAdOx1.HTI at D92 and D106 and with MVA.HTI at D120.

Ten mice (5 females, 5 males) from group 2b (G2b) were treated by two IM injections (one injection in each thigh of the mouse) of vehicle DNA.HTI buffer at D1, D8 and D15 and vehicle MVA.HTI buffer at D22 and D36. Then, all mice were treated with ChAdOx1.HTI buffer at D92 and D106. Finally, mice were treated with MVA.HTI buffer at D120.

Ten mice (5 females, 5 males) from group 3a (G3a) were treated by two IM injections (one injection in each thigh of the mouse) of ChAdOx1.HTI at D1 and D15.

Ten mice (5 females, 5 males) from group 3b (G3b) were treated by two IM injections (one injection in each thigh of the mouse) of ChAdOx1.HTI at D1 and D15. Then, all mice were treated with MVA.HTI at D29 and D43.

Ten mice (5 females, 5 males) from group 4 (G4) were treated by two IM injections (one injection in each thigh of the mouse) of ChAdOx1.HTI buffer at D1 and D15 and MVA.HTI buffer at D29 and D43.

The experimental design and treatment groups are summarized in Table 2 below.

TABLE 2
Experimental design

| | Number of | Administration | | | |
|---|---|---|---|---|---|
| | animals | route | Days | Treatment | Sacrifice |
| 1a | 10 (5♀/5♂) | IM | D1, D8, D15 | DNA.HTI | D50 |
| | | | D22, D36 | MVA.HTI | |
| 1b | 10 (5♀/5♂) | IM | D1, D8, D15 | DNA.HTI | D92 |
| | | | D22, D36 | MVA.HTI | |
| 1c | 10 (5♀/5♂) | IM | D1, D8, D15 | DNA.HTI | D134 |
| | | | D22, D36 | MVA.HTI | |
| | | | D92, D106 | ChAdOx1.HTI | |
| | | | D120 | MVA.HTI | |
| 2a | 10 (5♀/5♂) | IM | D1, D8, D15 | DNA.HTI buffer (PBS) | D134 |
| | | | D22, D36 | MVA.HTI buffer (Tris buffer) | |
| | | | D92, D106 | ChAdOx1.HTI | |
| | | | D120 | MVA.HTI | |
| 2b | 10 (5♀/5♂) | IM | D1, D8, D15 | DNA.HTI buffer (PBS) | D134 |
| | | | D22, D36 | MVA.HTI buffer (Tris buffer) | |
| | | | D92, D106 | ChAdOx1.HTI buffer (A438 | |
| | | | | buffer) | |
| | | | D120 | MVA.HTI buffer (Tris buffer) | |
| 3a | 10 (5♀/5♂) | IM | D1, D15 | ChAdOx1.HTI | D29 |
| 3b | 10 (5♀/5♂) | IM | D1, D15 | ChAdOx1.HTI | D57 |
| | | | D29, D43 | MVA.HTI | |
| 4 | 10 (5♀/5♂) | IM | D1, D15 | ChAdOx1.HTI buffer (A438 | D57 |
| | | | | buffer) | |
| | | | D29, D43 | MVA.HTI buffer (Tris buffer) | |

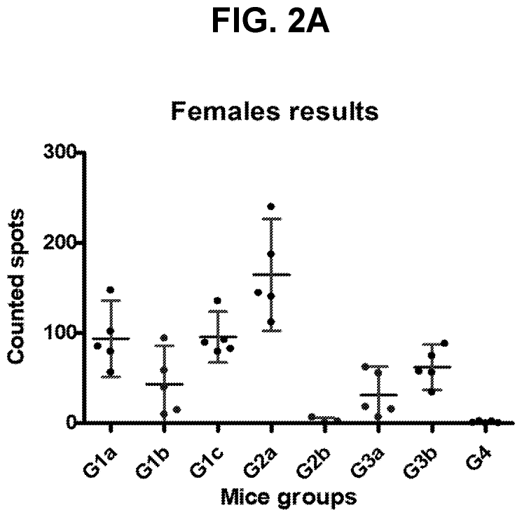

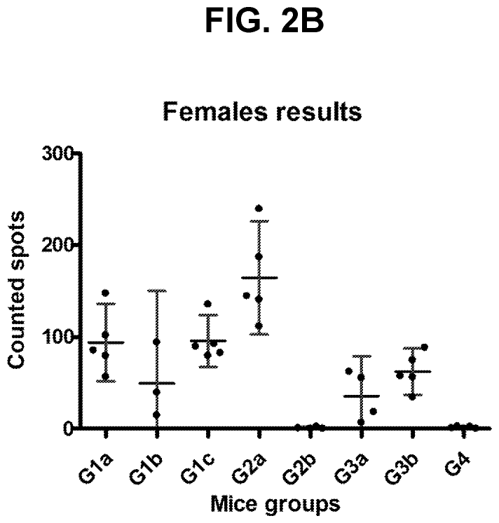

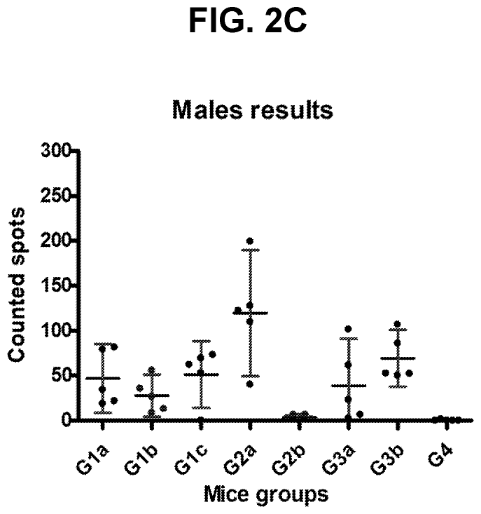

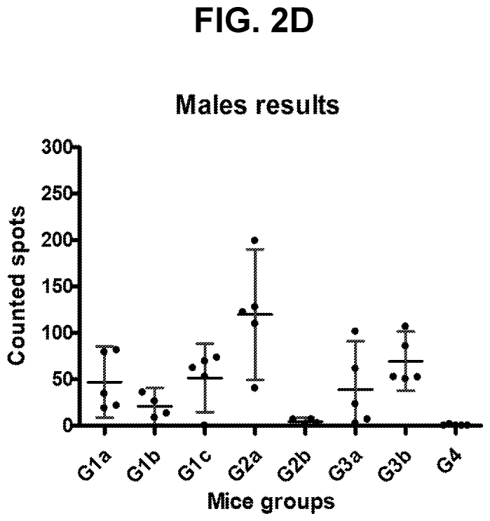

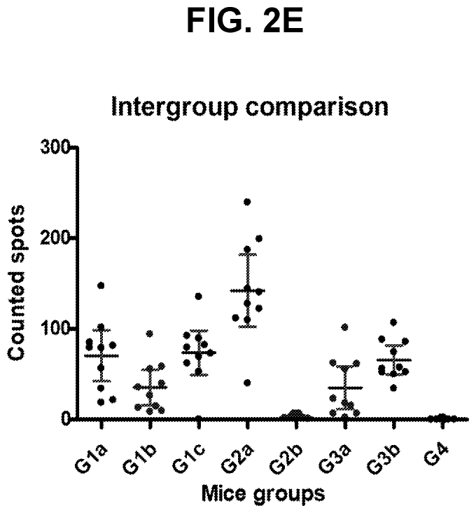

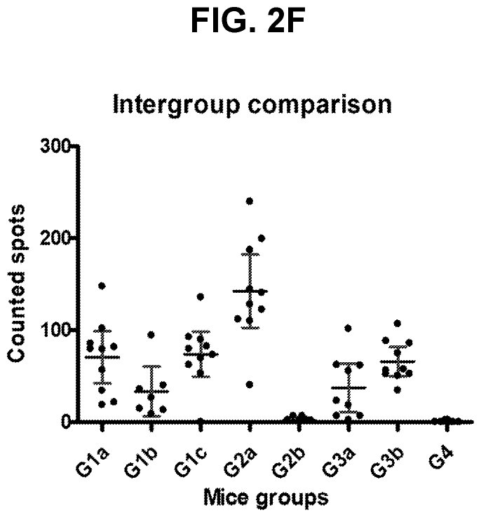

Immunogenicity was measured by ELISPOT analysis as described in the previous examples. Immunogenicity results are shown in FIGS. 2A-2F. The different regimes tested (DDDMM, DDDMMCCM, CCM, CC, CCMM) induced an immune response against the HTI immunogen.

### Example 6

Immune Responses of Combinations of DNA.HTI, MVA.HTI and ChAdOx1.HTI in C57BL/6 Mice

42 mice were randomized according to body weight criteria in 7 groups (6 mice per group). Test Substances and vehicle were prepared and administered by intramuscular injection (IM), as described above. The mice from group 1 were treated with PBS at day 1 (D1), D15, D29 and D43. The mice from group 2 were treated with DNA.HTI at D1, D15, D29 and then with MVA.HTI at D43. The mice from group 3 were treated with PBS at D1, and then with DNA.HTI at D15, D29 and D43. The mice from group 4 were treated with PBS at D1, and then with DNA.HTI at D15, D29 and D43. The mice from group 5 were treated with DNA.HTI at D1, D15 and D29, and then with MVA.HTI at D43. The mice from group 6 were treated with DNA.HTI at D1, D15 and D29, and then with MVA.HTI at D43. The mice from group 7 were treated with PBS at D1 and D15 and then with ChAdOx1.HTI at D29 and D43. Treatments are abbreviated herein as D=DNA.HTI; M=MVA.HTI; and C=control (vehicle or PBS). As such, DDDM represents three treatments of DNA.HTI followed by one treatment of MVA.HTI, for example.

The experimental design and treatment groups are summarized in Table 3 below.

TABLE 3
Experimental design

| | Number of | Administration | | | |
|---|---|---|---|---|---|
| Group | animals | route | Treatment schedule | Treatment | Sacrifice |
| 1 | 6 female | IM | D1, D15, D29, D43 | Vehicle (PBS) | D57 |
| 2 | | | 1. D1, D15, D29* | 1.DNA.HTI (Batch No. PB108) | |
| | | | 2. D43 | 2. MVA.HTI (Batch No. | |
| | | | | 0010915) | |
| 3 | | | 1. D1 | 1. Vehicle (PBS) | |
| | | | 2. D15, D29, D43 | 2. DNA.HTI (Batch No. | |
| | | | | PB125) | |
| 4 | | | 1. D1 | 1. Vehicle (PBS) | |
| | | | 2. D15, D29, D43 | 2. DNA.HTI (Batch No. | |
| | | | | PB136) | |
| 5 | | | 1. D1, D15, D29 | 1. DNA.HTI (Batch No. | |
| | | | 2. D43 | PB125) | |
| | | | | 2. MVA.HTI (Batch No. | |
| | | | | 0010216) | |
| 6 | | | 1. D1, D15, D29 | 1. DNA.HTI (Batch No. | |
| | | | 2. D43 | PB136) | |
| | | | | 2. MVA.HTI (Batch No. | |
| | | | | 0020518) | |
| 7 | | | 1. D1, D15 | 1. Vehicle (PBS) | |
| | | | 2. D29, D43 | 2. ChAdOx1.HTI (Batch No. | |
| | | | | H.0003) | |

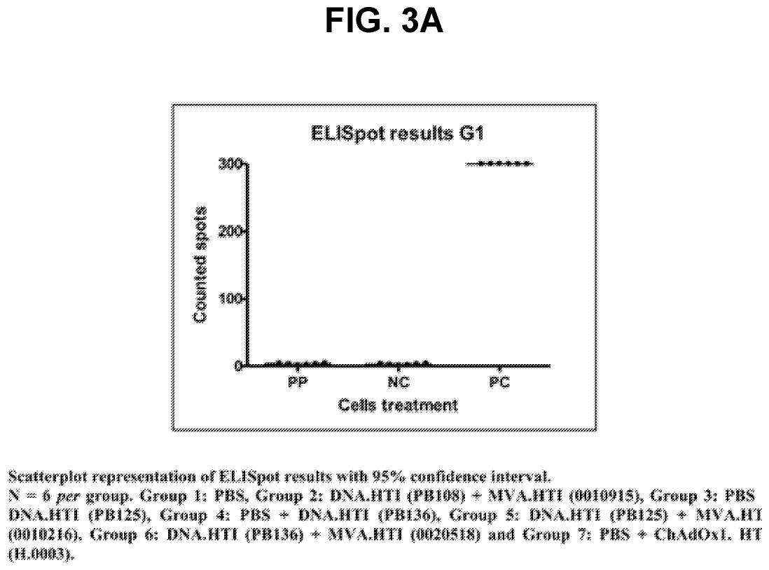

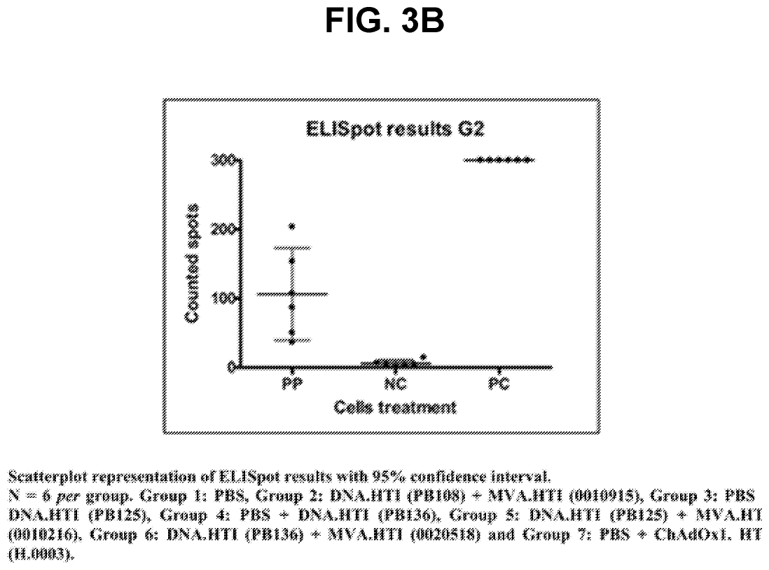

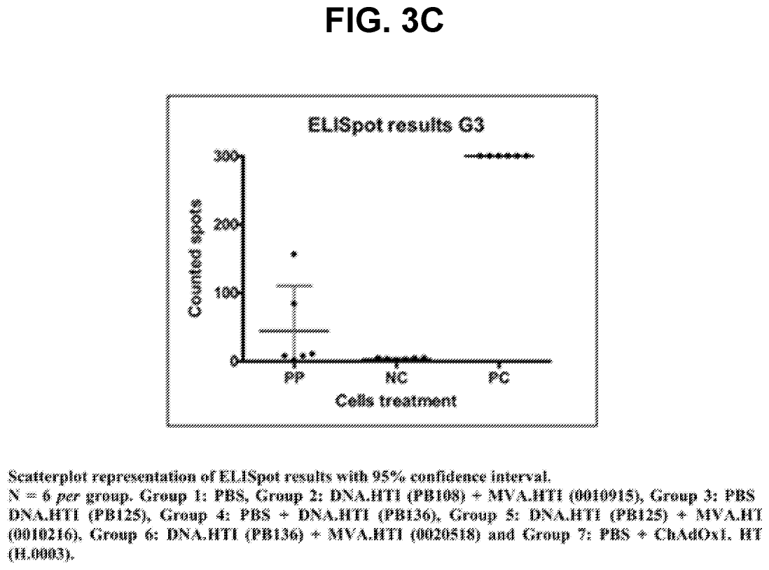

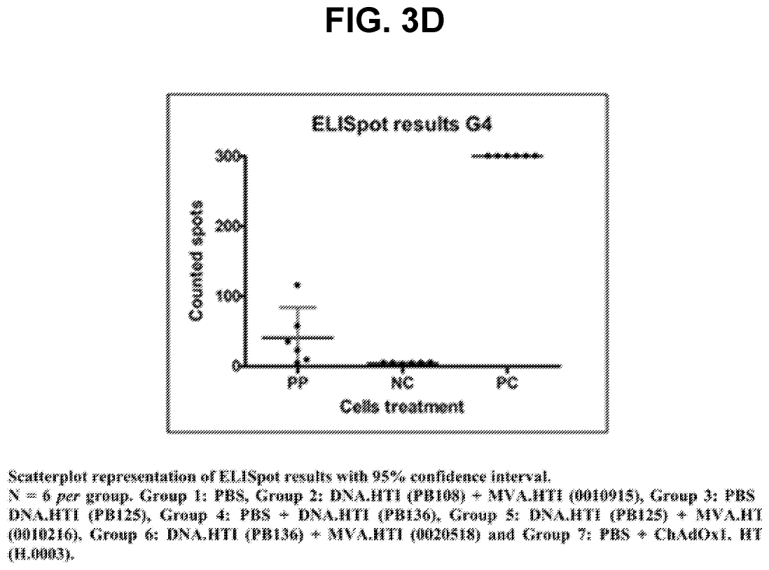

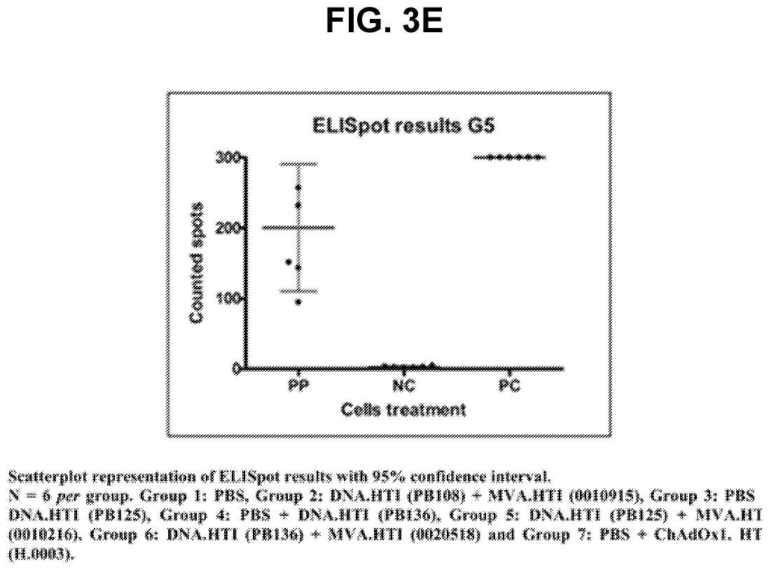

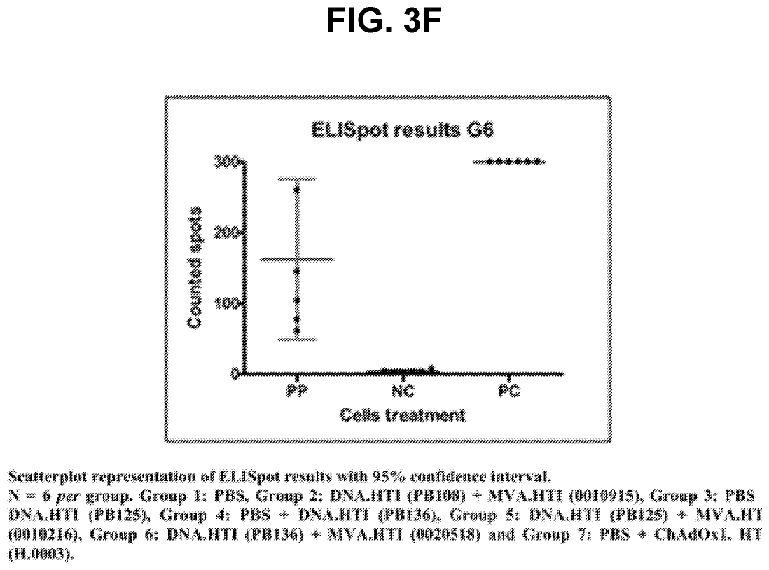

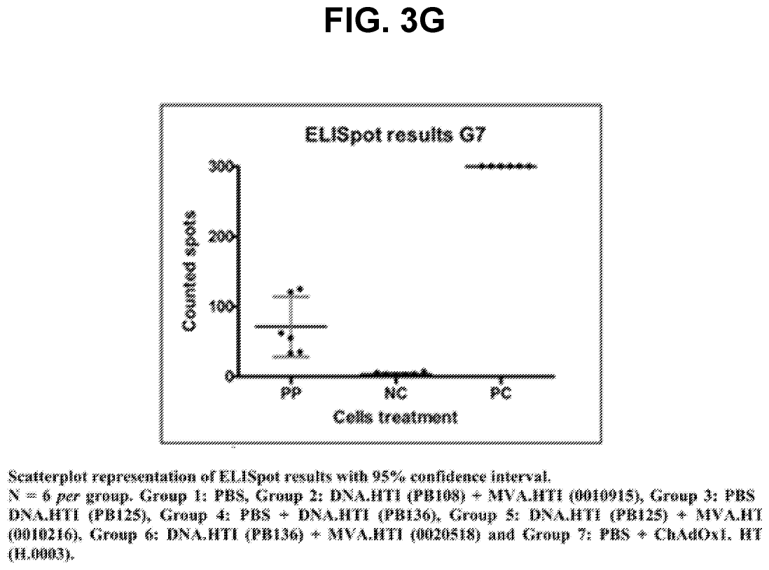

Immunogenicity was measured by ELISPOT analysis as described in the previous examples. Immunogenicity results are shown in FIGS. 3A-3G for each of groups 1-7, respectively. The different treatment regimes tested (DDDM, DDD, DMCM, and C) induced an immune response against the HTI immunogen.

### Example 7

Immunogenicity of Repeated Doses of ChAdOx1.HTI in in C57BL6 Mice

ChAdOx1.HTI was administered by intramuscular injection to all Group 2 animals on Day 1, 15 and 29, with control animals from Group 1 receiving the formulation buffer on the same days. The main and biodistribution study animals were sacrificed on Day 43 and the recovery phase animals were sacrificed four weeks later (Day 72). The experimental design and treatment groups are summarized in Table 4 below.

TABLE 4
Experimental design

| | | | Number of animals | | | | | |
|---|---|---|---|---|---|---|---|---|
| | | | | | Recovery | | Biodistribution | |
| | | Dose | Main study | | phase | | study | |
| Group | Treatment | (/mouse) | Male | Female | Male | Female | Male | Female |
| 1 | Control | 0 | 12 | 12 | 7 | 7 | 5 | 5 |
| 2 | ChAdOx1.HTI | 1 × 10¹⁰ vp | 12 | 12 | 7 | 7 | 5 | 5 |

For this assay, splenocytes, at 0.4×10⁶ cells/per well, were treated with the HTI positive peptides number 23 and peptide number 101 at 10 μg/mL, Concanavalin A (ConA; positive control at 0.5 μg/mL), anti-CD3 (a second positive control 0.01 μg/mL), or medium (background control) for approximately 18-20 hours on IFN-γ-coated membranes in microtitre plates. During this incubation period secreted IFN-γ was bound by the immobilized antibody in the immediate vicinity of the secreting cells. After washing away cells and residual antigen, a biotinylated monoclonal antibody specific for mouse IFN-γ was added to the wells. Following a wash to remove any unbound biotinylated antibody, alkaline phosphatase (ALP) conjugated to streptavidin was added. Unbound enzyme was subsequently removed by washing and a substrate solution (BCIP/NBT nitro-blue tetrazolium and 5-bromo-4-chloro-3′-indolyphosphate) was added. A blue-black colored precipitate formed at the sites of cytokine localization and appeared as spots, with each individual spot representing an individual IFN-γ secreting cell.

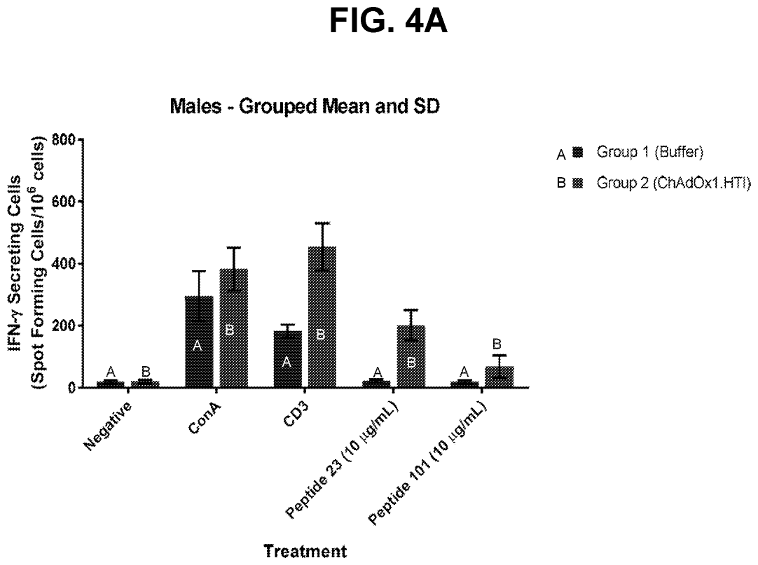

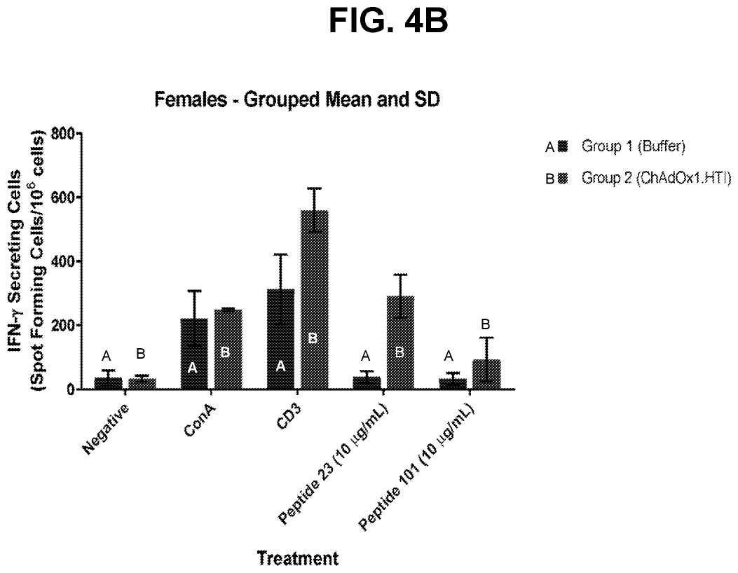

FIG. 4A-4B show the IFN-γ response of male and female treatment groups, respectively. A strong IFN-γ response was seen in the positive controls, ConA and CD3, for both males and females in both groups. Group 2 (ChAdOx1.HTI) animals for both sexes showed a higher response with CD3 than Group 1 (Buffer) animals. A low IFN-γ response was seen in the negative (media) control wells. There was an increase of around 10 fold for both males and females in the IFN-γ response for all Group 2 (ChAdOx1.HTI) animals compared to Group 1 (Buffer) animals in wells treated with peptide #23. Wells treated with peptide #101 resulted in a small increase of around 3 fold in the IFN-γ for all Group 2 animals compared to Group 1 animals for both males and females.

### Example 8

Prime-Boost Strategies in Mouse (C57BL/6 and BalbC)

Magnitude and breath of the response to different prime-boost strategies was measured in C57BL/6 females and BalbC males and females as in Table 5 below:

TABLE 5
Experimental design

| | Prime | Boost | Timing | | | | | Week | Week | Week |
|---|---|---|---|---|---|---|---|---|---|---|
| Experiment | (dose/animal) | (dose/animal) | Week 0 | Week 1 | Week 4 | Week 5 | Week 8 | 10 | 11 | 14 |
| M | 1xMVA.HTI | | Arrival | vac1 | sac1 | | | | | |
| | 10\hat{ }6 PFU | | | MVA.HTI | | | | | | |
| C | 1xChAd.HTI | | Arrival | vac1 | sac1 | | | | | |
| | 10\hat{ }9 VP | | | ChAd.HTI | | | | | | |
| CM | 1xChAd.HTI | 1xMVA.HTI | Arrival | vac1 | | vac2 | sac1 | | | |
| | 10\hat{ }9 VP | 10\hat{ }6 PFU | | ChAd.HTI | | MVA.HTI | | | | |
| DDD | 3xDNA.HTI | | Arrival | vac1 | | vac2 | vac3 | sac1 | | |
| | 100 ug | | | DNA.HTI | | DNA.HTI | DNA.HTI | | | |
| DDM | 3xDNA.HTI | 1xMVA.HTI | Arrival | vac1 | | vac2 | vac3 | | | sac1 |
| | 100 ug | 10\hat{ }6 PFU | | DNA.HTI | | DNA.HTI | DNA.HTI | | | |

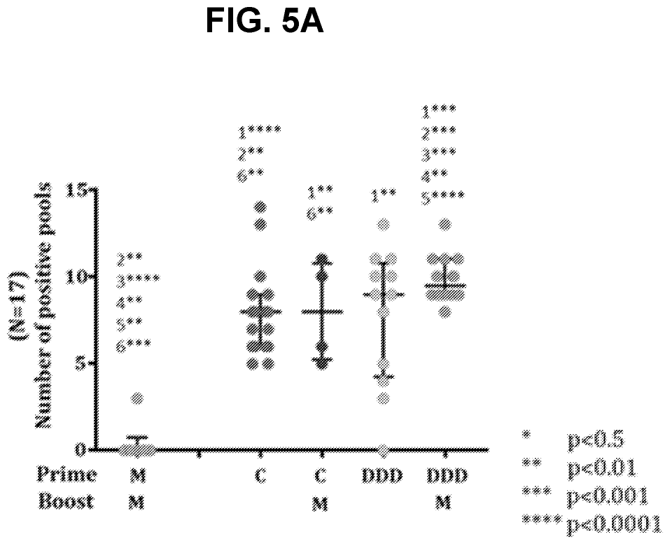

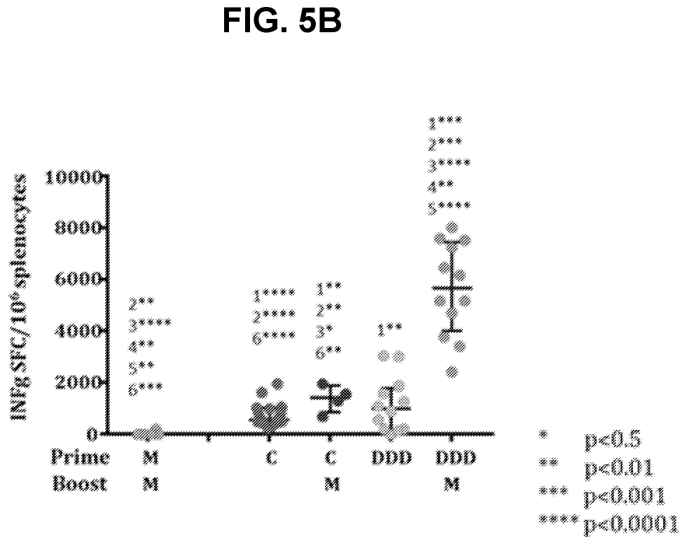

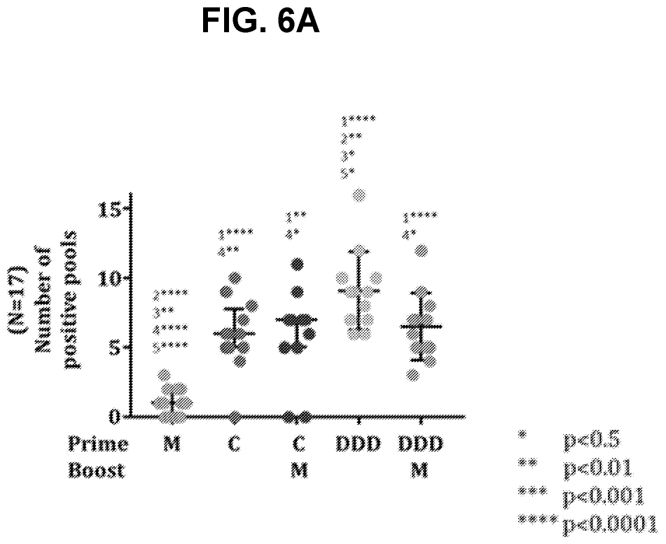

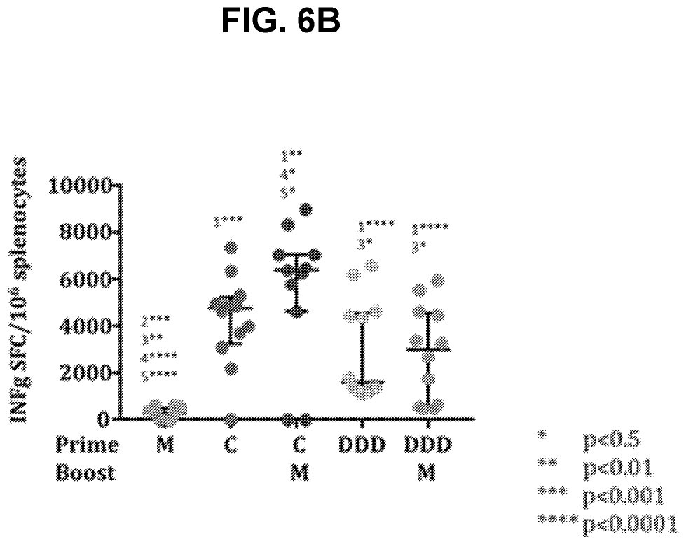

Results in C57BL/6 females are shown in FIG. 5A (number of positive pools) and FIG. 5B shows the INFγ spot forming colonies per 10⁶ splenocytes. Results in BalbC males and females are shown in FIG. 6A (number of positive pools) and FIG. 6B shows the INFγ spot forming colonies per 10⁶ splenocytes.

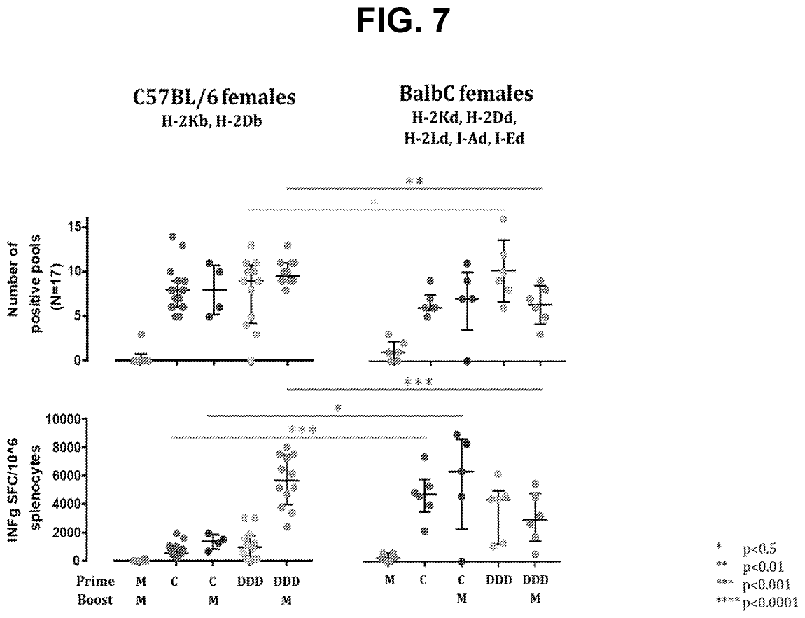

In addition, different prime-boost strategies were compared between the groups. These results are shown in FIG. 7.

### Example 9

Immune Response of Recombinant BCG Expressing HTI Prime and Recombinant ChAdOx1.HTI Boost in BALB/c Mice

The double auxotrophic E. coli-mycobacterial shuttle integrative vector, the p2auxo.int plasmid, contains the glyA and LysA genes which function as an antibiotic-free selection system in the auxotrophic strains of E. coli M15ΔglyA and BCG ΔLys, respectively. The synthetic sequence of HTI was codon optimized for BCG expression to match the G+C rich mycobacterial codon usage for enhanced expression. HTI G+C rich DNA sequence was synthesized by Geneart (USA). and ligated to the integrative p2auxo.int plasmid fused to the 19-kDa lipoprotein secretion signal sequence generating p2auxo.HTIint containing sites for integration into the BCG genome at the attB site. The ligation products were subsequently transformed into the E. coli M15ΔglyA strain for growth and selection.

Cells of the glycine auxotrophic strain of E. coli, M15ΔGly (Invitrogen), were cultured in minimal M9-derivative medium supplemented with glycine (70 μg/ml). The E. coli M15ΔGly cells were transformed with the p2auxo.HTIint plasmid by electroporation. The transformed cells were subsequently cultured on M9-D agar plates without glycine supplementation for selection or with glycine supplementation as a control.

The lysine auxotrophic BCG strain, BCGΔlys, was transformed with p2auxo.HTIint plasmid by electroporation. The mycobacteria were cultured in Middlebrook 7H9 broth medium or on Middlebrook agar 7H10 medium supplemented with albumin-dextrose-catalase (ADC; Difco) containing 0.05% Tween 80. L-lysine monohydrochloride (Sigma) was dissolved in distilled water and used as a supplement at a final concentration of 40 μg/ml. For transformation, BCG was cultured to an optical density of 1.5 at 600 nm, transformed using a Bio-Rad gene pulser electroporator at 2.5 kV, 25 g, and 1,000Ω. The transformants were then cultured on ADC-supplemented Middlebrook agar 7H10 medium containing 0.05% Tween 80 without lysine supplementation. The resulting colonies were assessed for plasmid insertion, integrity and HTI expression.

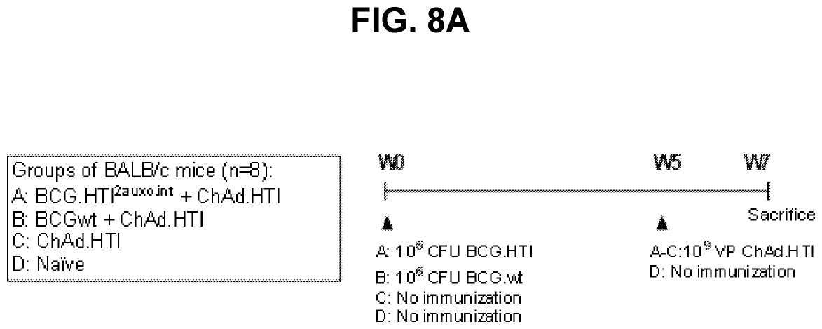

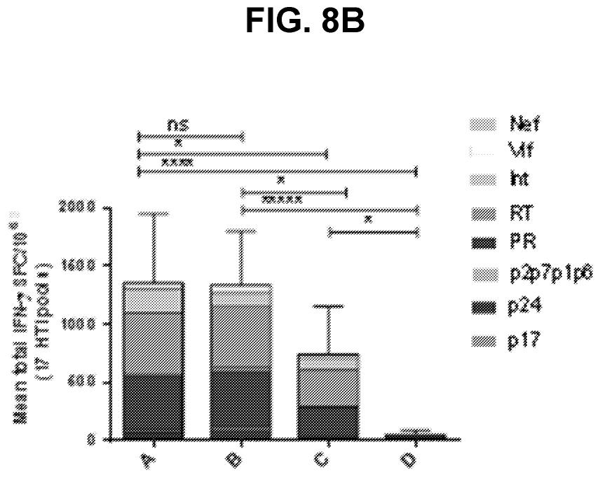

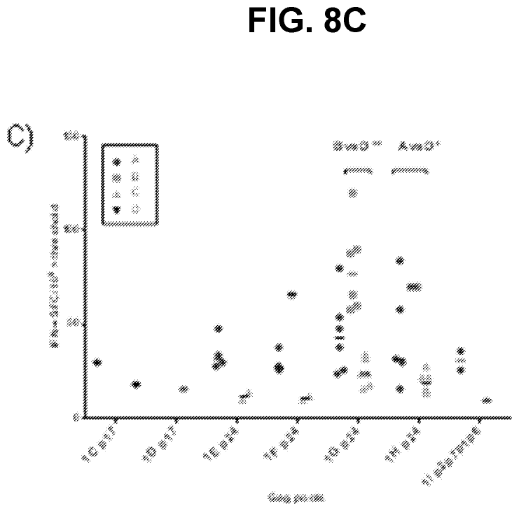

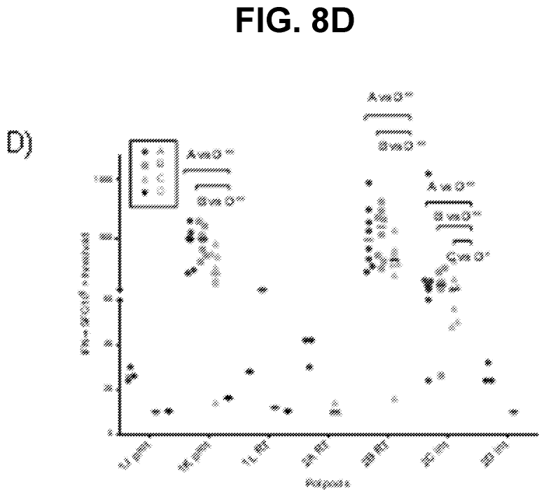

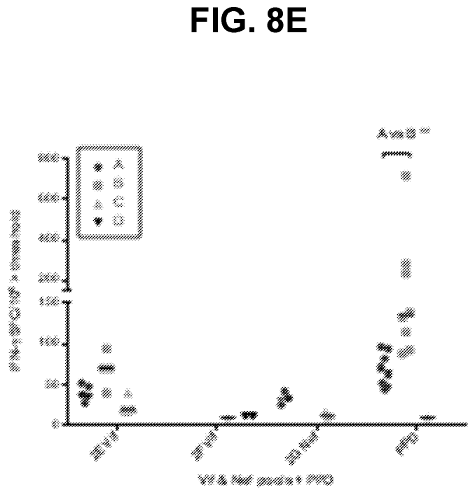

Groups of 8 adult (7-week-old) female BALB/c mice were immunized in one footpad and two groups were left unimmunized. Mice were treated as summarized in FIG. 8A. In particular, the first group received 10⁵ CFU of BCG.HTI2auxo.int (Group A), the second group received 10⁶ CFU BCG wt (Group B), both groups in one footpad. Two groups were left unimmunized (Groups C and D). After five weeks, groups A-C were boosted intramuscularly with 10⁹ vp of ChAdOx1.HTI and group D was left unimmunized. All mice were sacrificed two weeks after the boost for immunogenicity analyses. Immediately following sacrifice of the animals, splenocytes were harvested and homogenized using cell strainers (Falcon; Becton Dickinson) and 5-ml syringe rubber plungers. Red blood cells were removed with ACK lysing buffer (Lonza, Barcelona, Spain), and the splenocytes were washed and resuspended in complete medium (R10 (RPMI 1640 supplemented with 10% fetal calf serum and penicillin—streptomycin), 20 mmol/1 HEPES, and 15 mmol/12-mercaptoethanol).

The ELISPOT assay was performed using the commercial murine IFN-γ ELISPOT kit (Mabtech, Nacka Strand, Sweden) according to the manufacturer's instructions. For each animal, the mean of background responses was subtracted individually from all wells to allow comparison of IFN-γ spot forming cells/10⁶ between groups. To define positive responses a threshold was defined as at least 5 spots per well and responses exceeding the mean number of spots in negative control wells plus 3 standard deviations of the negative control wells.

### Example 10

Differential Recognition of Peptide Pools in BCG.HTI+ChAd.HTI Immunized BALB/c Mice

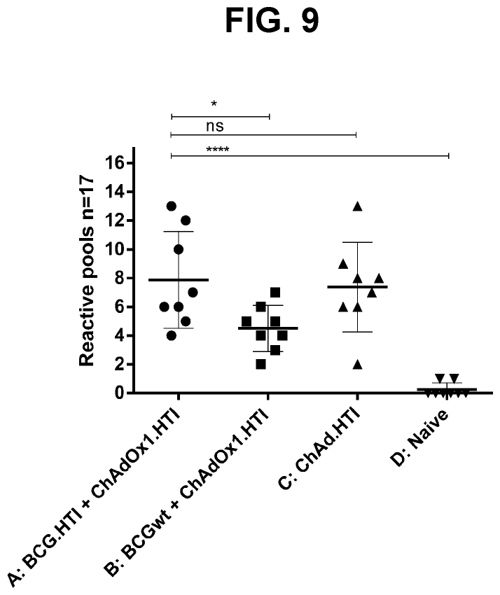

Adult mice (7-weeks-old, n=8/group) were immunized with either 105 cfu of BCG.HTI2auxo.int (id) and boosted with ChAdOx1.HTI (109 vp, im) after 5 weeks (Group A), or with 106 BCG.wt (id) and boosted with ChAdOx1.HTI (109 vp, im) after 5 weeks (Group B), or only immunized with ChAdOx1.HTI (109 vp, im) at week 5 (Group C), or left unimmunized (Group D). Two weeks post-boost, mice were sacrificed and splenocytes were isolated for ELISpot analysis and the numbers of reactive peptide pools (total n peptide pools=17) were compared for each mouse. Results are shown in FIG. 9.

ChAdOx1.HTI was immunogenic in terms of magnitude and breadth, both alone and primed with BCG.wt and with BCG.HTI2auxo.int. Both BCG.HTI2auxo.int primed mice and mice receiving ChAdOx1.HTI alone responded to an average of 7-8 peptide pools. Whereas mice primed with BCGwt alone only responded to an average of 4.5 peptide pools. This together suggests that, priming with BCG.HTI2auxo.int enhances the HTI specific immune response, when delivered with ChAdOx1.HTI, while maintaining the breadth of the response.

### Example 11

Clinical Efficacy of the DDDMM Priming Followed by CCM Boosting in HIV-1 Positive Individuals

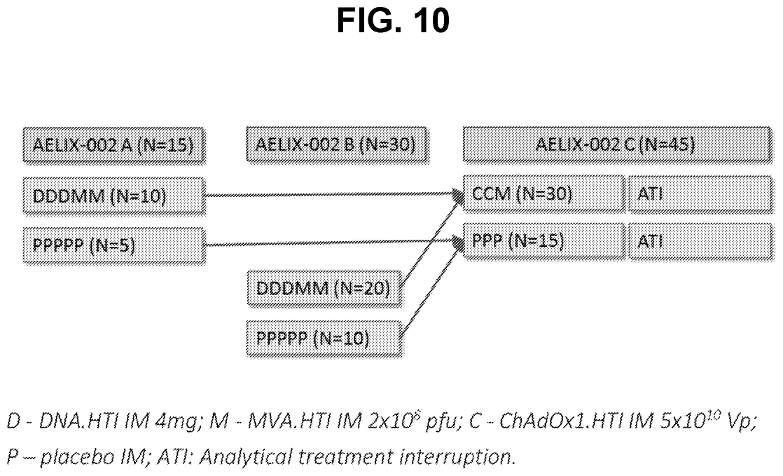

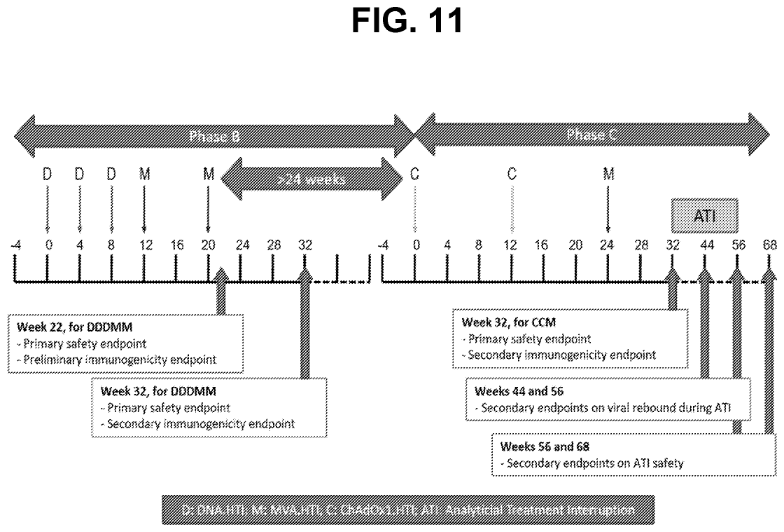

The DDDMM priming sequence, followed by CCM boosting is tested in HIV positive individuals, in a safety and immunogenicity trial entitled: A Phase I, Randomized, Double-Blind, Placebo-Controlled Safety, Tolerability and Immunogenicity Study of Candidate HIV-1 Vaccines DNA.HTI, MVA.HTI and ChAdOx1.HTI in Early Treated HIV-1 Positive Individuals (EUDRA-CT: 2017-000532-34). Briefly, the study recruits sequentially in 2 phases: Phase A (15 participants) and Phase B (30 participants) to evaluate safety, immunogenicity and efficacy of three novel HIV-1 vaccines administered in a heterologous prime-boost regimen DDDMM and CCM, followed by an Analytical Treatment Interruption (ATI) period (Phase C). The design of the study is shown in FIG. 10, and the design of the intervention is shown in FIG. 11. The products used in the trial are listed below in Table 6.

TABLE 6

| | | | Volume Injected |
|---|---|---|---|
| Vaccine/Placebo | Dosage | Formulation | (approximate) |
| DNA.HTI (D) | 4 mg | PBS pH 7.4 | 1 ml |
| | | | (500 μl × 2 injections) |
| MVA.HTI (M) | 2 × 10⁸ pfu | Tris pH 7.7 | 500 μl |
| | | | (250 μl × 2 injections) |
| ChAdOx1.HTI | 5 × 10¹⁰ Vp | L-Histidine: 10 mM NaCl: 35 mM | 500 μl |
| (C) | | Sucrose: 7.5% (w/v) MgCl2: 1 mM; | (250 μl × 2 injections) |
| | | EDTA disodium: 0.1 mM Tween 80 | |
| | | (Polysorbate-80): 0.1% (w/v) Ethanol | |
| | | 0.5%: (v/v) HCl: Adjusted to pH 6.6 | |
| Normal saline | 0.9% NaCl | | Matched to test |
| placebo | | | products |

Primary objectives are safety and tolerability. Secondary objectives are (1) to evaluate the immunogenicity of DNA.HTI, MVA.HTI and ChAdOx1.HTI vaccines as part of heterologous prime-boost regimens (DDDMM and CCM) in early treated HIV-1 positive individuals, and (2) to evaluate whether the heterologous prime-boost vaccination of DNA.HTI, MVA.HTI and ChAdOx1.HTI vaccines is able to prevent or delay viral rebound, induce post-rebound viral control, and/or prevent or delay the need for resumption of antiretroviral therapy during an analytical treatment interruption (ATI) of antiretroviral therapy in early treated HIV-1 positive individuals.

Secondary Endpoints are:

(1) T-Cell Immunogenicity:
Proportion of participants that develop de-novo T cell responses to HTI-encoded regions as determined by IFNγ ELISPOT assay in vaccine and placebo recipients.
Breadth and magnitude of total vaccine induced HIV-specific responses measured by IFNγ ELISPOT in vaccine and placebo recipients.

(2) Viral Rebound During an ATI Period (from Phase C Week 32 to Phase C Week 56)
Percentage of participants with viral remission, defined as plasma viral load (pVL)<50 copies/mL at 12 and 24 weeks after ATI (visits Phase C week 44 and week 56).
Percentage of participants with viral control, defined as a pVL <2,000 copies/mL at 12 and 24 weeks after ATI (visit Phase C week 44 and week 56).
Time to viral detection, defined as the time from ATI start (visit Phase C week 32) to first occurrence of detectable pVL (>50 copies/mL).
Time to viral rebound, defined as the time from ATI start (visit Phase C week 32) to first occurrence of pVL >10,000 copies/mL.
Percentage of participants who remain off cART at 12 and 24 weeks after ATI (visits Phase C week 44 and week 56).
Time off cART, defined as time to cART resumption since ATI start (visit Phase C week 32).

Main inclusion criteria are as follows:

1. Confirmed HIV-1 infection.

2. On combined antiretroviral treatment (defined as ≥3 antiretroviral drugs) initiated within 6 months of estimated time of HIV-1 acquisition.

3. Willing and able to be adherent to their cART regimen for the duration of the study.

4. Optimal virological suppression for at least 1 year defined as maintained pVL below the limit of detection (based on current available assays, 20, 40 or 50 copies/ml) allowing for isolated blips (<200 cop/ml, non-consecutive, representing <10% total determinations).

5. Being on the same cART regimen for at least 4 weeks at screening visit.

6. Nadir CD4 count ≥200 cells per mm3. Isolated lower counts at the moment of acute HIV-1 infection will be allowed only if appropriate immune recovery was followed after cART initiation (as is criteria 7).

7. Stable CD4 counts ≥500 cells per mm3 (Phase A) or ≥400 cells per mm3 (Phase B) for the last 6 months at screening visit.

Main exclusion criteria are as follows:

1. Pregnancy or lactating.

2. When available, pre-cART genotypic data that demonstrates the presence of clinically significant drug resistance mutations that would prevent the construction of a viable cART regimen post-treatment interruption.

3. Reported periods of suboptimal adherence to cART

4. History of past antiretroviral treatment interruptions longer than 2 weeks.

5. Participation in another clinical trial within 12 weeks of study entry (at screening visit).

6. Any AIDS-defining disease or progression of HIV-related disease.

7. History of autoimmune disease.

8. History or clinical manifestations of any physical or psychiatric disorder which could impair the subject's ability to complete the study.

9. Receipt of approved vaccines within 2 weeks of study entry and along the duration of the trial.

### Example 12

Clinical Efficacy of the CC Priming Followed by MM Boosting in HIV-1 Positive Individuals

The CC priming sequence, followed by MM boosting is tested in HIV positive individuals, in a safety and immunogenicity trial (EUDRA-CT: 2018-002125-30). Briefly, out of 90 participants in the study, 60 patients are randomized to other study arms, 10 patients are randomized to receive CCMM, and 20 patients are randomized to receive placebo. Primary objective is safety and tolerability. Secondary objectives are:

1. Viral Rebound
To evaluate whether CCMM is able to prevent or delay viral rebound, induce post-rebound viral control, and/or prevent or delay the need for resumption of antiretroviral therapy (ART) following an ATI in early treated HIV-1 infection.

2. T Cell Immunogenicity
To evaluate the immunogenicity of CCMM in early treated HIV-1 infection.

Clinical endpoints of such objectives are defined as follows:

1. For Viral Rebound
Proportion of participants with control of viral load below detectable level 12 and 24 weeks after start of ART interruption (remission).
Proportion of participants with control of viral load <2000 copies/mL 12 and 24 weeks after start of ART interruption (viral control).
Proportion of participants that remain off ART 12 and 24 weeks after start of ART interruption.
Time to viral rebound (first confirmed detectable plasma viral load [pVL]≥50 copies/mL) during ATI.
Time to viral rebound (first confirmed pVL value >10,000 copies/mL) during ATI.
Time to ART resumption after ART interruption.

2. For T Cell Immunogenicity
Proportion of participants with de-novo T cell responses to HTI encoded regions during the 48-week treatment period as determined by IFNγ enzyme-linked immunospot (ELISPOT) assay in vaccine and placebo recipients.
Breadth and magnitude of total vaccine-induced HIV-1 specific responses during the 48 week treatment period measured by IFNγ ELISPOT in vaccine and placebo recipients.

The ChAdOx1.HTI and MVA.HTI vaccines are administered as 2 intramuscular (IM) injections, 1 injection into the deltoid region of each arm. Each IM injection of ChAdOx1.HTI will deliver 0.5 mL of vaccine, for a total daily dose of 1 mL. Each IM injection of MVA.HTI will deliver 0.5 mL of vaccine, for a total daily dose of 1 mL. The vaccine regimens are as follows:

1. CCMM:
ChAdOx1.HTI at Week 0 and Week 12 (2 total doses of 5×10¹⁰ vp)
MVA.HTI at Week 24 and Week 36 (2 total doses of 2×10⁸ pfu each)

2. Placebo:
ChAdOx1.HTI placebo at Week 0 and Week 12 (2 total doses)
MVA.HTI placebo at Week 24 and Week 36 (2 total doses)

The study is conducted in 3 periods. In Period 1 (Week −4 to Week 48), participants have a screening visit within 28 days before the first dose of the IMP. Participants are randomly assigned to treatment at Visit 1 (Week 0) and receive the first vaccine or matched vaccine placebo administration on the same day. Participants continue to take their ART during Period 1. Participants receive up to 2 doses of ChAdOx1.HTI, and 2 doses of MVA.HTI or matching placebos over the minimum 48-week treatment period. Participants in Period 1 who prematurely discontinue study treatment complete the Week 84/Early Termination assessments. In Period 2 (Week 48 to Week 72), all participants discontinue ART after the Week 48 visit. Participants are monitored for rebound in HIV-1 plasma viremia for 24 weeks of close observation and follow-up. For the 24 weeks of ATI, participants return for weekly visits. Participants have their ART restarted during the ATI if specific criteria are met.

Participants who complete 24 weeks of ATI without restarting ART continue into Period 3; these participants resume ART at Week 72 and are followed for an additional 12 weeks. Participants who restart their ART during Period 2 complete the Week 84/Early Termination assessments. If a participant withdraws from the study during Period 2 and has not restarted ART, the investigator decides on the timing for restarting ART and the Week 84/Early Termination assessments are performed.

Inclusion criteria on the trial are:

1. Understands the study information provided and is capable of giving written informed consent, in the opinion of the investigator or designee.

2. Has confirmed HIV-1 infection.

3. Is receiving ART, defined as ≥3 antiretroviral drugs, that was initiated within 6 months of the estimated date of HIV-1 acquisition.

4. Has been virologically suppressed, defined pVL <50 copies/mL, for at least 1 year before the screening visit; isolated blips allowed (<200 copies/mL, nonconsecutive, representing <10% of total determinations).

5. Has been on the same ART regimen for at least 4 weeks before the screening visit.

6. Has stable CD4 counts ≥450 cells/mm³ for the 6 months before the screening visit.

7. Has nadir CD4 count ≥200 cells/mm³; isolated lower counts at the moment of acute HIV-1 infection will be allowed only if appropriate immune recovery was followed after ART initiation (see inclusion criterion #6).

8. Is ≥18 and <61 years of age on the day of the screening visit.

Exclusion criteria are:

1. Is pregnant or lactating at the screening visit or at any time during the study or is planning on becoming pregnant over the duration of the study.

2. Has pre-ART genotypic data, if available, that demonstrate the presence of clinically significant mutations that would prevent the construction of a viable ART regimen post-treatment interruption.

3. Has reported periods of suboptimal adherence to ART, defined as reported episodes of 72 hours without ART that were unrelated to participation in an ATI clinical study.

4. Has history of past known ATIs longer than 2 weeks.

5. Has participated in another interventional clinical study within 30 days before the screening visit.

6. Has any AIDS-defining disease or progression of HIV-related disease within 90 days of the screening visit or at randomization (i.e., Week 0, the day of first IMP dose).

7. Has a history of any moderate and/or severe autoimmune disease.

8. Has a history or clinical manifestations of any physical or psychiatric disorder that could impair the participant's ability to complete the study.

9. Has received approved vaccines within 2 weeks of study entry or will receive vaccines over the duration of the study without sponsor approval.

What is claimed is:

1. A method of treating or preventing a human immunodeficiency virus (HIV) infection or a disease associated with an HIV infection in a subject in need thereof, comprising:
(a) administering to the subject 1 to 10 administrations of a DNA vector encoding an immunogenic polypeptide, followed by 1 to 10 administrations of a first viral vector encoding the immunogenic polypeptide; and
(b) administering to the subject 1 to 10 administrations of a second viral vector encoding the immunogenic polypeptide;
wherein the immunogenic polypeptide comprises:
(i) a sequence having at least 90% identity to the sequence of SEQ ID NO:1,
(ii) a sequence having at least 90% identity to the sequence of SEQ ID NO:2,
(iii) a sequence having at least 90% identity to the sequence of SEQ ID NO:3,
(iv) a sequence having at least 90% identity to the sequence of SEQ ID NO:4,
(v) a sequence having at least 90% identity to the sequence of SEQ ID NO:5,
(vi) a sequence having at least 90% identity to the sequence of SEQ ID NO:6,
(vii) a sequence having at least 90% identity to the sequence of SEQ ID NO:7,
(viii) a sequence having at least 90% identity to the sequence of SEQ ID NO:8,
(ix) a sequence having at least 90% identity to the sequence of SEQ ID NO:9,
(x) a sequence having at least 90% identity to the sequence of SEQ ID NO:10,
(xi) a sequence having at least 90% identity to the sequence of SEQ ID NO:11,
(xii) a sequence having at least 90% identity to the sequence of SEQ ID NO:12,
(xiii) a sequence having at least 90% identity to the sequence of SEQ ID NO:13,
(xiv) a sequence having at least 90% identity to the sequence of SEQ ID NO:14,
(xv) a sequence having at least 90% identity to the sequence of SEQ ID NO:15, and
(xvi) a sequence having at least 90% identity to the sequence of SEQ ID NO:16.

2. The method of claim 1, wherein (a) comprises administering to the subject 1 to 4 administrations of the DNA vector encoding the immunogenic polypeptide, followed by 1 to 4 administrations of the first viral vector encoding the immunogenic polypeptide; and/or (b) comprises administering to the subject 1 to 4 administrations of the second viral vector encoding the immunogenic polypeptide.

3. The method of claim 1 or 2, wherein (a) comprises administering to the subject 3 administrations of the DNA vector encoding the immunogenic polypeptide, followed by 2 administrations of the first viral vector encoding the immunogenic polypeptide.

4. The method of any one of claims 1-3, wherein (b) comprises administering to the subject 2 administrations of the second viral vector encoding the immunogenic polypeptide, followed by 1 administration of the first viral vector encoding the immunogenic polypeptide.

5. The method of any one of claims 1-4, wherein the DNA vector comprises a human cytomegalovirus (CMV) promoter and/or a bovine growth hormone (BGH) polyadenylation site.

6. The method of any one of claims 1-5, wherein the first and/or second viral vector is a Modified Vaccinia Ankara (MVA) virus vector and/or a chimpanzee adenovirus (ChAd) vector.

7. The method of any one of claims 1-6, wherein (a) comprises administering to the subject 3 administrations of the DNA vector encoding the immunogenic polypeptide, followed by 2 administrations of an MVA vector encoding the immunogenic polypeptide; and (b) comprises administering to the subject 2 administrations of a ChAd vector encoding the immunogenic polypeptide, followed by 1 administration of a MVA vector encoding the immunogenic polypeptide.

8. A method of treating or preventing a HIV infection or a disease associated with an HIV infection in a subject in need thereof, comprising:
(a) administering to the subject 1 to 5 administrations of a first viral vector encoding the immunogenic polypeptide; and
(b) administering to the subject 1 to 5 administrations of a second viral vector encoding the immunogenic polypeptide;
wherein the immunogenic polypeptide comprises:
(i) a sequence having at least 90% identity to the sequence of SEQ ID NO:1,
(ii) a sequence having at least 90% identity to the sequence of SEQ ID NO:2,
(iii) a sequence having at least 90% identity to the sequence of SEQ ID NO:3,
(iv) a sequence having at least 90% identity to the sequence of SEQ ID NO:4,
(v) a sequence having at least 90% identity to the sequence of SEQ ID NO:5,
(vi) a sequence having at least 90% identity to the sequence of SEQ ID NO:6,
(vii) a sequence having at least 90% identity to the sequence of SEQ ID NO:7,
(viii) a sequence having at least 90% identity to the sequence of SEQ ID NO:8,
(ix) a sequence having at least 90% identity to the sequence of SEQ ID NO:9,
(x) a sequence having at least 90% identity to the sequence of SEQ ID NO:10,
(xi) a sequence having at least 90% identity to the sequence of SEQ ID NO:11,
(xii) a sequence having at least 90% identity to the sequence of SEQ ID NO:12,
(xiii) a sequence having at least 90% identity to the sequence of SEQ ID NO:13,
(xiv) a sequence having at least 90% identity to the sequence of SEQ ID NO:14,
(xv) a sequence having at least 90% identity to the sequence of SEQ ID NO:15, and
(xvi) a sequence having at least 90% identity to the sequence of SEQ ID NO:16.

9. The method of claim 8, wherein (a) comprises administering to the subject 2 administrations of the first viral vector encoding the immunogenic polypeptide.

10. The method of claim 8 or 9, wherein (b) comprises administering to the subject 2 administrations of the second viral vector encoding the immunogenic polypeptide.

11. The method of any one of claims 8-10, wherein the first viral vector is a ChAd vector and/or the second viral vector is an MVA vector.

12. The method of any one of claims 8-11, wherein (a) comprises administering to the subject 2 administrations of a ChAd vector encoding the immunogenic polypeptide; and (b) comprises administering to the subject 2 administrations of an MVA vector encoding the immunogenic polypeptide.

13. The method of any one of claims 1-7, wherein the DNA vector is administered at a dose of from about 0.1 mg to about 20 mg.

14. The method of claim 13, wherein the DNA vector is administered at a dose of from about 0.5 mg to about 10 mg.

15. The method of claim 13, wherein the DNA vector is administered at a dose of from about 1 mg to about 8 mg.

16. The method of claim 13, wherein the DNA vector is administered at a dose of about 4 mg.

17. The method of any one of claims 1-16, wherein the first and/or second viral vector is administered at a dose of from about 1×10⁷ plaque forming units (pfu) to about 1×10⁹ pfu.

18. The method of claim 17, wherein the first and/or second viral vector is administered at a dose of from about 5×10⁷ pfu to about 5×10⁸ pfu.

19. The method of claim 17, wherein the first and/or second viral vector is administered at a dose of about 2.5×10⁸ pfu.

20. The method of any one of claims 1-16, wherein the first and/or second viral vector is administered at a dose of from about 1×10⁹ viral particles to about 5×10¹¹ viral particles.

21. The method of claim 20, wherein the first and/or second viral vector is administered at a dose of from about 1×10¹⁰ to about 1×10¹¹ viral particles.

22. The method of claim 20, wherein the first and/or second viral vector is administered at a dose of about 5×10¹⁰ viral particles.

23. The method of any one of claims 1-22, wherein each administration is separated by a period of from about 15 days to about 18 months.

24. The method of any one of claims 1-22, wherein each administration is separated by a period of from about 1 week to about 24 months.

25. The method of any one of claims 1-22, wherein each administration is separated by a period of from about 2 weeks to about 56 weeks.

26. The method of any one of claims 1-22, wherein each administration is separated by a period of from about 4 weeks to about 12 weeks.

27. The method of any one of claims 1-26, wherein the administering of (a) is separated from the administering of (b) by a period of from about 2 months to about 24 months.

28. The method of claim 27, wherein the administering of (a) is separated from the administering of (b) by a period of from about 3 months to about 18 months.

29. The method of any one of claims 1-7, wherein (a) comprises administering to the subject:
(i) 3 administrations of the DNA vector encoding the immunogenic polypeptide, each separated by a period of about 4 weeks;
(ii) 1 administration of the first viral vector encoding the immunogenic polypeptide about 4 weeks after (a)(i); and
(iii) 1 administration of the first viral vector encoding the immunogenic polypeptide about 8 weeks after (a)(ii); and (b) comprises administering to the subject:
(i) 2 administrations the second viral vector encoding the immunogenic polypeptide, each separated by a period of about 12 weeks; and
(ii) 1 administration of the first viral vector encoding the immunogenic polypeptide about 12 weeks after (b)(i);
wherein the administering of (b) is separated from the administering of (a) by a period of about 24 weeks.

30. The method of claim 29, wherein the administrations of (a)(i) are at a dose of about 4 mg, the administration of (a)(ii) is at a dose of about 2×10⁸ pfu, the administration of (a)(iii) is at a dose of about 2×10⁸ pfu, the administrations of (b)(i) are at a dose of about 5×10¹⁰ viral particles, and/or the administration of (b)(ii) is at a dose of about 2×10⁸ pfu.

31. The method of claim 29 or 30, wherein the DNA vector of (a)(i) comprises a human cytomegalovirus (CMV) promoter and/or a bovine growth hormone (BGH) polyadenylation site.

32. The method of any one of claims 29-31, wherein the first viral vector is an MVA vector.

33. The method of any one of claims 29-32, wherein the second viral vector is a ChAd vector.

34. The method of any one of claims 29-33, wherein the first viral vector is an MVA vector and the second viral vector is a ChAd vector.

35. The method of any one of claims 8-12, wherein (a) comprises administering to the subject 2 administrations of the first viral vector encoding the immunogenic polypeptide, each separated by a period of about 12 weeks; and (b) comprises administering to the subject 2 administrations the second viral vector encoding the immunogenic polypeptide, each separated by a period of about 12 weeks; and wherein the administering of (b) is separated from the administering of (a) by a period of about 12 weeks.

36. The method of claim 35, wherein the administrations of (a) are at a dose of about 5×10′° viral particles, and/or the administrations of (b) are at a dose of about 2×10⁸ pfu.

37. The method of claim 35 or 36, wherein the first viral vector is a ChAd vector.

38. The method of any one of claims 35-37, wherein the second viral vector is an MVA vector.

39. The method of any one of claims 35-38, wherein the first viral vector is a ChAd vector and the second viral vector is an MVA vector.

40. The method of any one of claims 1-39, wherein the immunogenic polypeptide comprises:
(i) a sequence having at least 95% identity to the sequence of SEQ ID NO:1,
(ii) a sequence having at least 95% identity to the sequence of SEQ ID NO:2,
(iii) a sequence having at least 95% identity to the sequence of SEQ ID NO:3,
(iv) a sequence having at least 95% identity to the sequence of SEQ ID NO:4,
(v) a sequence having at least 95% identity to the sequence of SEQ ID NO:5,
(vi) a sequence having at least 95% identity to the sequence of SEQ ID NO:6,
(vii) a sequence having at least 95% identity to the sequence of SEQ ID NO:7,
(viii) a sequence having at least 95% identity to the sequence of SEQ ID NO:8,
(ix) a sequence having at least 95% identity to the sequence of SEQ ID NO:9,
(x) a sequence having at least 95% identity to the sequence of SEQ ID NO:10,
(xi) a sequence having at least 95% identity to the sequence of SEQ ID NO:11,
(xii) a sequence having at least 95% identity to the sequence of SEQ ID NO:12,
(xiii) a sequence having at least 95% identity to the sequence of SEQ ID NO:13,
(xiv) a sequence having at least 95% identity to the sequence of SEQ ID NO:14,
(xv) a sequence having at least 95% identity to the sequence of SEQ ID NO:15, and
(xvi) a sequence having at least 95% identity to the sequence of SEQ ID NO:16.

41. The method of claim 40, wherein the immunogenic polypeptide comprises the sequences of SEQ ID NOs:1-16.

42. The method of any one of claims 1-41, wherein at least two of the sequences of (i)-(xvi) are adjoined by an amino acid linker.

43. The method of claim 42, wherein the amino acid linker is a single, dual, or triple alanine linker, and wherein the linker results in the formation of an AAA sequence in the junction region between adjoining sequences, and/or wherein the sequence of each of (i) to (xvi) is 11-85 amino acids in length.

44. The method of any one of claims 1-43, wherein the immunogenic polypeptide further comprises a signal peptide at the N-terminus of the immunogenic polypeptide.

45. The method of any one of claims 1-43, wherein the immunogenic polypeptide comprises the sequence of SEQ ID NO:99 or wherein the immunogenic polypeptide is encoded by a nucleic acid comprising the SEQ ID NO:100 or 101.

46. The method of any one of claims 1-45, wherein the disease associated with an HIV infection is an acquired immune deficiency syndrome (AIDS), AIDS-related complex (ARC), or HIV opportunistic disease.

47. The method of any one of claims 1-46, wherein the HIV is HIV type 1 (HIV-1).

48. The method of any one of claims 1-46, wherein the HIV is HIV type 2 (HIV-2).

49. The method of any one of claims 1-48, wherein the subject is a human subject.
